(19) United States
(12) Patent Application Publication
(10) Pub. No.: US 2014/0178962 A1
(43) Pub. Date: Jun. 26, 2014

(54) ENGINEERED CARBONIC ANHYDRASE PROTEINS FOR CO2 SCRUBBING APPLICATIONS

(71) Applicants: Francis Raymond Salemme, Yardley, PA (US); Patricia C. Weber, Yardley, PA (US)

(72) Inventors: Francis Raymond Salemme, Yardley, PA (US); Patricia C. Weber, Yardley, PA (US)

(73) Assignee: Imiplex LLC, Yardley, PA (US)

(21) Appl. No.: 13/797,283

(22) Filed: Mar. 12, 2013

Related U.S. Application Data
(60) Provisional application No. 61/611,205, filed on Mar. 15, 2012.

(51) Int. Cl. C12N 9/96; C12N 9/88

(52) U.S. Cl. CPC C12N 9/96; C12N 9/88
USPC 435/177; 435/232; 435/188

(57) ABSTRACT

Engineered protein constructs with carbonic anhydrase catalytic activity, and their application in CO₂ scrubbing.

### APPLICATIONS

This application claims the benefit of U.S. Provisional Application No. 61/611,205, filed Mar. 15, 2012.

### SEQUENCE LISTING

The instant application contains a Sequence Listing which has been submitted in ASCII format via EFS-Web and is hereby incorporated by reference in its entirety. Said ASCII copy, created on Oct. 9, 2013, is named 85213345835SL.txt and is 97,143 bytes in size.

### FIELD OF THE INVENTION

Embodiments of the inventions include, for example, engineered structures of thermostable carbonic anhydrase and immobilized assemblies for CO₂ scrubbing applications.

### BACKGROUND OF THE INVENTION

Carbonic anhydrase enzymes are widely found in nature and catalyze the reversible interconversion of CO₂ and bicarbonate with high efficiency.

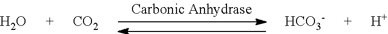

Carbonic anhydrase (CA) enzymes offer potential in systems designed to scrub CO₂ from closed atmospheric environments and/or industrial exhaust streams (Ge et al. 2002). Generally, thermostable enzymes derived from organisms that live in extreme environments are preferred for industrial applications. Thermostable enzymes offer isolation efficiencies when expressed in heterologous expressions systems like E. coli and are generally more resistant to denaturation effects that degrade enzyme activity in end-use applications.

The present invention describes novel engineered forms of gamma-CA enzymes (gCA) that are derived from thermophilic organisms. Owing to the unusual thermal stability and unique structural features of thermophilic gCA enzymes, they can be modified using protein engineering methods to produce novel protein compositions that meet key requirements for practical CO2 scrubbing systems that incorporate immobilized CA enzymes as the key catalytic element.

Although the use of thermostable CA enzymes for CO₂ scrubbing has been considered elsewhere (Borchart & Saunders 2010, Trachtenberg 2008), the proposed implementations had several limitations that impede their practical use in CO₂ scrubbing applications. The first limitation involves the relatively limited thermostability of the proteins identified. The second involves the method of enzyme immobilization. Lack of a suitably specific method of immobilization requires either the use of nonselective, harsh chemical methods, or imbedding in polymer matrices for enzyme immobilization. Both of these non-selective methods of immobilization destroy enzyme activity. In addition to the requirement for methods that can immobilize CA enzymes with minimal damage, reversible immobilization methods are desired, since it is anticipated that the active enzyme catalyst used in various configurations of CO₂ scrubbing apparatus will have to be replaced from time to time to account for eventual enzyme degradation in the end use apparatus application. Reversible enzyme binding is required since even thermostable enzymes are expected to become damaged through chemical oxidation of amino acids, amino acid deamidation, or other forms of chemical damage occurring while the enzyme is carrying out its catalytic conversion process. As in the case of most industrial catalysts, the effective lifetime of the catalyst will be shorter than the useful lifetime of the supporting mechanical apparatus, so requiring the ability to economically recharge the apparatus with catalyst at periodic intervals. Consequently, a practical system using CA enzymes as catalytic agents requires the utilization of CA enzymes having maximum thermal stability that can also be immobilized with high affinity using methods that both preserve enzyme activity and are reversible to allow the charge of enzyme catalyst in the apparatus to be periodically recycled with high efficiency. In the present invention we describe engineered forms of highly thermostable CA enzymes that incorporate several features required for practical CO₂ scrubbing applications, including 1) low production cost and ease of isolation, 2) high catalytic turnover rate, 3) useful lifetime and stability in the integrated apparatus, and 4) ability to be reversibly immobilized on the reactor substrate to allow apparatus recharging.

In an embodiment of the invention as described herein, a two-dimensional (2D) nanostructure includes a proteinaceous hexagonal tessellation on a fluid layer coated on a substrate. The proteinaceous hexagonal tessellation can include two or more trimer nodes bound to two or more struts. The trimer nodes can include an amino acid subsequence greater than 90% identical to a subsequent coding for a gamma carbonic anhydrase enzyme. Each trimer node can have C3 symmetry and include three (3) subunits forming a single polypeptide chain having a terminus. Each subunit of each trimer node can have a specific binding site including a pair of bound biotin or biotin derivative groups. The terminus of the single polypeptide chain of the trimer node can include a polyhistidine. Each strut can include a streptavidin or streptavidin derivative including pairs of biotin binding sites. Each trimer node and each strut can be bound by the biotin or biotin derivative groups of the trimer node specific binding site being bound with a pair of biotin binding sites of the strut. The fluid layer can include a metal chelate. The polyhistidine can be bound to the metal chelate.

The metal chelate can be, for example, a nickel chelate, Ni-NTA (nickel nitrilotriacetic acid, also termed nickel-nitrolo acetic acid), a metal chelate phospholipid, and/or a nickel chelate phospholipid. The fluid layer can include a lipid and/or a phospholipid bilayer. The fluid layer can include Ni-NTA-DOGA (nickel-2-(biscarboxymethyl-amino)-6-[2-(1,3)-di-O-oleyl-glyceroxy)-acetyl-amino]hexanoic acid) and/or dioleoyl phosphatidylcholine. The substrate can include a polymer, polyethylene glycol (PEG), a metal coating, a gold coating, a tethered cholesterol, a ceramic, and/or a glass.

The trimer node can be engineered from a thermophilic microorganism, for example, through recombinant techniques including molecular cloning. The trimer node can have a stable tertiary and/or quaternary structure at a temperature of about 30° C., 40° C., 50° C., 60° C., 70° C., 80° C., 90° C., 100° C., 110° C., 120° C., or greater.

The trimer node can include an amino acid sequence of carbonic anhydrase Methanosarcina thermophila (pdb code 1thj), carbonic anhydrase Pyrococcus horikoshii OT3 (pdb code 1v3w), carboxysomal gamma-carbonic anhydrase CcmM (pdb code 3kwc), or an alternative gamma-carbonic anhydrase identified by amino acid sequence homology with the proteins listed above.

The specific binding site can include a pair of bound biotin groups, a pair of bound iminobiotin groups, or a combination of a bound biotin group and a bound iminobiotin group. The polyhistidine can be a histidine 6-mer (HHHHHH (SEQ ID NO: 1)). The strut can include a streptavidin including two pairs of biotin binding sites.

The proteinaceous hexagonal tessellation can extend in a given direction regularly for at least about 100 nm, 200 nm, 500 nm, 1000 nm, 2000 nm, or 5000 nm. The proteinaceous hexagonal tessellation can extend regularly in a direction for at least about 2, 4, 10, 20, 40, or 100 hexagonal cells.

### SUMMARY OF THE INVENTION

A thermostable, trimeric gCA composition incorporating specific features for surface immobilization.

A thermostable, single-chain gCA composition incorporating specific features for surface immobilization and formation of trivalent linkages with streptavidin.

A thermostable, single-chain gCA composition incorporating specific features for surface immobilization and formation of bivalent linkages with streptavidin.

A hyperthermostable, trimeric gCA composition incorporating specific features for surface immobilization.

A hyperthermostable, trimeric gCA composition incorporating specific features for surface immobilization and formation of trivalent linkages with streptavidin.

A hyperthermostable, single-chain gCA composition incorporating specific features for surface immobilization.

A hyperthermostable, single-chain gCA composition incorporating specific features for surface immobilization and formation of a monovalent linkage with streptavidin.

A hyperthermostable, single-chain gCA composition incorporating a specific terminal sequence for enzymatic biotinylation.

Trimeric thermostable gCA compositions incorporating terminal sequences for surface immobilization.

Single-chain thermostable gCA compositions incorporating terminal sequences for surface immobilization.

An embodiment wherein a trimeric gGA construct having three pairs of biotin binding sites forms a complex with three streptavidin tetramers, producing an assembly with six biotin binding sites in a trigonal arrangement.

An embodiment wherein a single-chain gGA construct having three pairs of biotin binding sites forms a complex with three streptavidin tetramers, producing an assembly with six biotin binding sites in a trigonal arrangement.

An embodiment wherein two single-chain, terminally biotinylated, gCA constructs are immobilized on surfaces through links to surface-bound streptavidin tetramers.

An embodiment wherein a trimeric gGA construct having three pairs of biotin binding sites forms a complex with three avidin tetramers, producing an assembly with six biotin binding sites in a trigonal arrangement.

An embodiment wherein a single-chain gGA construct having three pairs of biotin binding sites forms a complex with three avidin tetramers, producing an assembly with six biotin binding sites in a trigonal arrangement.

An embodiment wherein two single-chain, terminally biotinylated, gCA constructs are immobilized on surfaces through links to surface-bound avidin tetramers.

In an embodiment, an engineered gamma carbonic anhydrase enzyme (gCA) polypeptide can include residues 1-213 of Table 1, Sequence 1 (SEQ ID NO: 8) or a sequence greater than 90% identical thereto, residues 1-173 of Table 1, Sequence 4 (SEQ ID NO: 11) or a sequence greater than 90% identical thereto, or residues 1-181 of Table 1, Sequence 5 (SEQ ID NO: 12) or a sequence greater than 90% identical thereto. The engineered gCA polypeptide can have the sequence of Table 1, Sequence 1 (SEQ ID NO: 8), sequence of Table 1, Sequence 2 (SEQ ID NO: 9), sequence of Table 1, Sequence 3 (SEQ ID NO: 10), sequence of Table 1, Sequence 4 (SEQ ID NO: 11), sequence of Table 1, Sequence 5 (SEQ ID NO: 12), sequence of Table 1, Sequence 6 (SEQ ID NO: 13), sequence of Table 1, Sequence 7 (SEQ ID NO: 14), or sequence of Table 1, Sequence 8 (SEQ ID NO: 15), or a sequence greater than 90% identical to any of these.

An embodiment of an engineered gCA polypeptide can include a polypeptide sequence of the form A(BDBD)ᵥBC. v can be 0 or 1. A can be a sequence of Amino Terminus Sequence List A that is no amino acid, HₙXₘ, with X any amino acid and m ranging from 0 to 20 and n ranging from 0 to 7 or from 4 to 7 (SEQ ID NO: 52), or LERAPGGLNDIFEAQKIEWHEXᵣ (SEQ ID NO: 49), with each amino acid of the Xᵣ subsequence independently selected as any amino acid and r ranging from 0 to 7 or from 4 to 7. B can be a sequence of Sequence List B that is selected from the group consisting of SEQUENCES 9 through 41 of Table 2. C can be a sequence of Carboxy Terminus Sequence List C that is no amino acid, XₚH_{q}, with X any amino acid and p ranging from 0 to 20 and q ranging from 0 to 7 or from 4 to 7 (SEQ ID NO: 53), or XₛLERAPGGLNDIFEAQKIEWHE (SEQ ID NO: 50), with each amino acid of the Xₛ subsequence independently selected as any amino acid and s ranging from 0 to 7 or from 4 to 7. D can be a sequence of Sequence List D that is GₐS_{b}G_{c}S_{d} (SEQ ID NO: 51), with a, b, c, and d each independently ranging from 0 to 4. An embodiment of a trimeric gCA construct can include a first engineered gCA polypeptide, a second engineered gCA polypeptide, and a third engineered gCA polypeptide, each having a sequence of form ABC. The first engineered gCA polypeptide can be bound through a zinc atom to the second engineered gCA polypeptide, the second engineered gCA polypeptide can be bound through a zinc atom to the third engineered gCA polypeptide, and the third engineered gCA polypeptide can be bound through a zinc atom to the first engineered gCA polypeptide. An embodiment of a trimeric trigonal scaffold unit, can include a trimeric gCA construct, with each engineered gCA polypeptide including a specific binding site comprising a pair of bound biotin or biotin derivative groups and three streptavidin tetramers, with each streptavidin tetramer having a top pair of biotin binding sites and a bottom pair of biotin binding sites. The pair of bound biotin or biotin derivative groups of each engineered gCA polypeptide can be bound to the top pair of biotin binding sites of the streptavidin tetramer, so that the bottom pairs of biotin binding sites of the three streptavidin tetramers are in a trigonal arrangement. An avidin tetramer can be substituted for the streptavidin tetramer. A single chain gCA construct can have a sequence of form ABDBDBC. An embodiment of a single chain trigonal scaffold unit can include a single chain gCA construct, with each B sequence of the engineered gCA polypeptide including a specific binding site comprising a pair of bound biotin or biotin derivative groups and three streptavidin tetramers, with each streptavidin tetramer having a top pair of biotin binding sites and a bottom pair of biotin binding sites. The pair of bound biotin or biotin derivative groups of each B sequence of the engineered gCA polypeptide can be bound to the top pair of biotin binding sites of the streptavidin tetramer, so that the bottom pairs of biotin binding sites of the three streptavidin tetramers are in a trigonal arrangement. A single chain trigonal scaffold unit can have the specific binding site including a pair of cysteine substitutions, the bound biotin or biotin derivative group being bound to the cysteine substitution, and the pair of bound biotin or biotin derivative groups being located complimentary to a pair of biotin binding sites on streptavidin. A di-biotin linked 2D hexagonal lattice can include multiple single chain trigonal scaffold units. Each single chain trigonal scaffold unit can be connected to another single chain trigonal scaffold unit by a pair of bi-functional crosslinking agents. Each bi-functional crosslinking agent can include two binding groups. Each binding group of the bi-functional crosslinking agent can bind to the bottom pair of biotin binding sites in the streptavidin. The binding group can be biotin, a biotin derivative, desthiobiotin, iminobiotin, HABA (4′-hydroxyazobenzene-2-carboxylic acid), a HABA derivative, or an amino acid sequence comprising WSHPNFEK (SEQ ID NO: 54) or a sequence about 90% or greater identical thereto. A surface immobilized protein construct can include a first engineered gCA polypeptide having a biotin group covalently bonded to a sequence inserted at or near its amino terminus or carboxy terminus, a second engineered gCA polypeptide having a biotin group covalently bonded to a sequence inserted at or near its amino terminus or carboxy terminus, and a streptavidin tetramer having a first top and a second top biotin binding site and a first bottom and a second bottom biotin binding site. Two biotin groups can be bound to a surface. The biotin group of the first engineered gCA polypeptide can be bound to the first top biotin binding site of the streptavidin tetramer. The biotin group of the second engineered gCA polypeptide can be bound to the second top biotin binding site of the streptavidin tetramer. The first bottom and second bottom biotin binding sites can be bound to the two biotin groups bound to the surface. A single chain gCA construct can have sequence A as HₙXₘ (SEQ ID NO: 52), optionally bound to a metal, or LERAPGGLNDIFEAQKIEWHEXᵣ (SEQ ID NO: 49) and can have sequence C as XₚH_{q} (SEQ ID NO: 53), optionally bound to a metal, or XₛLERAPGGLNDIFEAQKIEWHE (SEQ ID NO: 50).

An embodiment of a two-dimensional nanostructure includes a proteinaceous hexagonal tessellation and/or a di-biotin linked 2D hexagonal lattice on a fluid layer coated on a substrate. The proteinaceous hexagonal tessellation can include a plurality of trimer nodes bound to a plurality of struts. Each trimer node can have C3 symmetry and comprises 3 subunits forming a single polypeptide chain having a terminus. Each single chain gCA construct can have a terminus. Each subunit of each trimer node can have a specific binding site comprising a pair of bound biotin or biotin derivative groups. The terminus of the single polypeptide chain of the trimer node can include a polyhistidine. The terminus of a single chain of the single chain gCA construct can include a polyhistidine. Each strut can include a streptavidin or streptavidin derivative comprising pairs of biotin binding sites. Each trimer node and each strut can be bound by the biotin or biotin derivative groups of the trimer node specific binding site being bound with a pair of biotin binding sites of the strut. The fluid layer can include a metal chelate. The polyhistidine can be bound to the metal chelate. The single polypeptide chain of the trimer node can include a subsequence greater than 90% identical to a subsequent coding for a gamma carbonic anhydrase enzyme. The single chain gCA construct can have a stable tertiary structure at a temperature of about 70° C. or greater.

A method includes introducing a nucleotide sequence coding for an engineered gCA amino acid sequence having an Amino Terminal Biotinylation Sequence or a Carboxy Terminus Biotinylation Sequence into a host organism (for example, E. coli). The host organism can be cultured. The host organism can be lysed to release the engineered gCA amino acid sequence into a first solution. The first solution can be contacted with a substrate functionalized with a form of avidin at a first pH, so that the biotinylated gCA amino acid sequence binds to the avidin. The substrate with the avidin can be contacted with a second solution at a second pH, so that the avidin releases the biotinylated gCA amino acid sequence in a purified form. For example, engineered or modified avidin can exhibit strong biotin binding at about pH 4 and release biotin at about pH of 10 or greater. An Amino Terminal Biotinylation Sequence can be LERAPGGLNDIFEAQKIEWHEXᵣ (SEQ ID NO: 49), wherein each amino acid of the Xᵣ subsequence is independently selected as any amino acid and r ranges from 0 to 7 or from 4 to 7. A Carboxy Terminal Biotinylation Sequence can be XₛLERAPGGLNDIFEAQKIEWHE (SEQ ID NO: 50), with each amino acid of the Xₛ subsequence independently selected as any amino acid and s ranging from 0 to 7 or from 4 to 7. Other engineered or modified avidins exhibiting strong biotin binding at about pH 7, 6, 5, 4, 3, 2, 1, 0 or less and exhibiting release of biotin at about pH 7, 8, 9, 10, 11, 12, 13, 14 or greater can be used. Alternatively, streptavidin can be used instead of avidin, and contacted with deionized water at about 70 deg C. to release the biotin.

### BRIEF DESCRIPTION OF THE DRAWINGS

Table 1. A list of sequences of engineered forms of gCA based on core structures derived from the Methanosarcina thermophila and Pyrococcus horikoshii gCA enzymes.

Table 2. A list of thermophilic gCA sequences suitable as core structures for engineered gCA constructs useful in CO₂ scrubbing applications.

FIGS. 1A through 1B: Schematic CO₂ scrubbing apparatus. FIG. 1A shows that a gas stream 101 containing CO₂ is admitted to a chamber 102 that is divided by an asymmetric semipermeable membrane 103. The semipermeable membrane 103 is exposed to the gas stream environment 104 on one side of the semipermeable membrane, and to a liquid carrier environment 105 on the other side. Carbonic anhydrase (CA) enzyme molecules immobilized on the liquid-exposed side of the semipermeable membrane 103 catalyze the conversion of CO₂ diffusing across the membrane into bicarbonate anion that dissolves in the liquid phase contained in the volume 105. A pump 106 moves the bicarbonate-enriched liquid into a second chamber 107 that is divided by a second asymmetric semipermeable membrane 108. The membrane, incorporating surface-bound carbonic anhydrase enzyme molecules, catalyzes the conversion of bicarbonate anion present in the liquid chamber 109 into CO₂, which diffuses across the membrane 108 into the gas-containing chamber 110 where the gas can exhaust or otherwise be removed. A second pump 111 optionally assists in recirculating the bicarbonate transfer fluid between chambers 102 and 107. FIG. 1B shows an alternative embodiment of engineered CA enzymes 113 immobilized on the surface of resin particles or other bead materials 112 that are suitable for packing in beds or columns incorporated in CO₂ scrubbing apparatus.

FIGS. 2A through 2C: Gamma carbonic anhydrase (gCA) structure. FIG. 2A shows a projection down the C3 symmetry axis of the trimeric gamma carbonic anhydrase isolated from the thermophilic microorganism Methanosarcina thermophila (www.rcsb.org pdb code 1thj). The label 201 designates one of the catalytic zinc atoms of the timer that is ligated to 3 histidine residues. FIG. 2B shows a projection down the C3 symmetry axis of the trimeric gamma carbonic anhydrase isolated from the hyperthermophile Pyrococcus horikoshii OT3 (www.rcsb.org pdb code 1v3w). The label 202 designates one of the catalytic zinc atoms of the trimer that is ligated to 3 histidine residues. FIG. 2C shows a side view of the backbone ribbon structure of Pyrococcus horikoshii OT3 (www.rcsb.org pdb code 1v3w) gamma carbonic anhydrase trimer. The label 203 designates one of the catalytic zinc atoms of the trimer.

FIGS. 3A through 3B: Schematic architecture of gCA proteins engineered for reversible immobilization on surfaces. FIG. 3A shows a symmetric trimer composed of identical subunits 301, 302, and 303. An active site zinc atom 304 is located at each subunit interface. Each subunit sequence can be modified through addition of an immobilization sequence at either the amino terminus 305 or carboxy terminus 306 of the subunit polypeptide chain. FIG. 3B shows a single-chain construct where individual subunit chains 308, 309, 310 have been linked into a single polypeptide chain with linkers 312 and 313. The single-chain structure can be additionally modified through incorporation of an immobilization sequence at either the amino terminus 311 or carboxy terminus 314 of the continuous polypeptide chain.

FIGS. 4A through 4B: Molecular architecture of gamma carbonic anhydrase proteins engineered for reversible immobilization on surfaces. FIG. 4A shows a backbone side view of an engineered form of a trimeric 1v3w gCA (γCA, gamma carbonic anhydrase). The active site zinc of one subunit is shown as 401. The polypeptide chain C-terminus of each subunit has been extended with a poly-His terminal sequence 402 that enables binding the trimer to a Ni-NTA functionalized surface. FIG. 4B shows a backbone side view of an engineered form of the 1v3w gCA where the trimer has been engineered as a single-chain construct through the introduction of two subunit linkers 403. The C-terminus helix 404 of the single-chain construct has been extended with a substrate sequence that allows the specific enzymatic addition of a covalently bound biotin group 405. Analogous structures exist for the 1thj gCA enzyme.

FIGS. 5A through 5B: Schematic of engineered gCA enzymes on CO₂ reaction membrane. FIG. 5A shows a schematic model of the 1v3w gCA extended-terminus timer 501 bound to a porous membrane substrate 502. Each enzyme trimer is bound to the membrane through 3 chemical linkages 503 formed between the membrane and the protein trimer. FIG. 5B shows a schematic model of the 1v3w biotinylated single-chain gCA 504 bound to a porous membrane substrate 505 through and intermediate streptavidin tetramer 506. The structure is formed by first immobilizing streptavidin to surface biotinylation sites 507.

FIGS. 6A through 6B: Biotinylated gCA single-chain constructs immobilized by streptavidin. FIG. 6A shows a ribbon model of two single-chain biotin-linked gCAs 601 (also FIG. 4B) bound to a surface-immobilized streptavidin tetramer 602. The streptavidin is immobilized by two surface bound biotin groups that can bind a pair of biotin-binding sites 603 on the streptavidin tetramer. FIG. 6B shows a molecular surface representation of the complex showing the position of the surface immobilization sites 604. FIG. 5B shows the assembly immobilized on a surface.

FIGS. 7A through 7D: Schematic architecture of gamma carbonic anhydrase proteins engineered for nanostructure formation. FIG. 7A shows a symmetric trimer composed of identical subunits where each subunit has been modified to incorporate 2 covalently bound biotin groups 701. The trimer can consequently for a trivalent interaction with three streptavidin tetramers. FIG. 7B shows a single-chain construct where three pairs of biotinylation sites have been incorporated in the single-chain construct to produce a trivalent node able to bind two streptavidin tetramers. FIG. 7C shows a single-chain construct where two pairs of biotinylation sites, 702 and 703, have been incorporated in the single-chain construct to produce a bivalent node able to bind two streptavidin tetramers. FIG. 7D shows a single-chain construct where a single pair of biotinylation sites, 704, have been incorporated in the single-chain construct to produce a monovalent node able to bind a single streptavidin tetramer.

FIGS. 8A through 8B: Molecular structure of a trigonal scaffold composed of a biotin substituted trimeric gCA complexed with 3 streptavidin tetramers. FIG. 8A shows a backbone ribbon representation of the 1v3w gCA trimer 801, where each subunit has been modified to incorporate 2 covalently bound biotin groups that allow binding to a streptavidin tetramer 802. FIG. 8B shows a molecular surface representation of the complex of FIG. 8A, indicating the projected positions of the biotin residues 803 that interconnect the central node with the peripherally bound streptavidin tetramers.

FIGS. 9A through 9B: Hexagonal pattern gCA nanostructure assembly. FIG. 9A outlines an efficient process of gCA hexagonal lattice nanostructure assembly. A trivalent trimeric gCA construct pre-saturated with three streptavidin tetramers to form the complex 901 is combined with free trimeric gCA 902 to form the hexagonal lattice 903. FIG. 9B outlines an efficient process of gCA hexagon nanostructure assembly. A bivalent single-chain gCA construct pre-saturated with two streptavidin tetramers to form the complex 904 is combined with free bivalent single chain gCA construct 905 to form the closed hexagon 906.

FIG. 10. Trigonal pattern gCA nanostructure assembly. The trivalent gCA node 1001 is combined with 3 streptavidin tetramers 1002 to form the trigonal scaffold 1003. The trigonal scaffold 1003 can be combined with the terminally biotinylated single-chain gCA construct 1004 to form the trigonal gCA nanoassembly 1005. Alternately, the trigonal scaffold 1003 can be combined with the monovalent, di-biotinylated single-chain gCA construct 1006 to form the trigonal gCA nanoassembly 1007.

FIGS. 11A through 11D: Trigonal nanoassembly surface packing. FIG. 11A shows a molecular model of the trigonal nanoassembly based on the 1v3w gCA molecular structure incorporating a central trivalent gCA construct, three linking streptavidin tetramers, and six terminally biotinylated single-chain gCA constructs. FIG. 11B illustrates that the nanoassembly of FIG. 11A can efficiently tie a 2D surface. FIG. 11C shows a molecular model of the trigonal nanoassembly based on the 1v3w gCA molecular structure incorporating a central trivalent gCA construct, three linking streptavidin tetramers, and three monovalent single-chain gCA constructs. FIG. 11D illustrates that the nanoassembly of FIG. 11C can efficiently tile a 2D surface.

FIGS. 12A through 12C: Expression Vectors: Vector constructions used for expression of engineered forms of gCA in E. coli. FIG. 12A shows the EXP14Q3193C2 vector expressing a trimeric, trivalent construct of the 1thj gCA from Methanosarcina thermophila. FIG. 12B shows the EXP14Q3193C3 vector expressing a single-chain, trivalent construct of the 1thj gCA from Methanosarcina thermophila. FIG. 12C shows the EXP14Q3193C4 vector expressing a single-chain, bivalent construct of the 1thj gCA from Methanosarcina thermophila.

FIGS. 13A through 13H: Nanostructure assembly on monolayers. FIG. 13 A shows a vessel 1301 containing an aqueous solution, on the surface of which is formed a monolayer consisting of a mixture of lipids 1302 and lesser amount of lipids 1303 that are functionalized on their head group with a Ni-NTA group. FIG. 13B illustrates the introduction of a trivalent node shown in plan 1304 and side view 1305. The trivalent node incorporates 3 pair of biotinylation sites 1306, and a terminal poly-Histidine sequence 1307. A solution of the node is introduced below the surface of the monolayer using a syringe 1308. The nodes 1309 attach to the Ni-NTA lipids through interactions formed between the Ni-NTA and the poly-Histidine terminus of the node. The monolayer is fluid, so that the nodes 1309 are free to diffuse in the plane of the monolayer. FIG. 13C shows the introduction of streptavidin 1310 under the surface of the monolayer using syringe 1311. Attachments formed between the freely diffusing nodes and streptavidin produce the assembled nanostructure 1312. FIG. 13D shows the assembled nanostructure and monolayer 1313 contacted by a surface 1312 with and affinity for the hydrophobic surface of the monolayer. FIG. 13E shows the assembled nanostructure and monolayer lifted from the liquid and attached to the surface 1314. FIG. 13F shows a schematic of a hexagonal nanolattice formed using streptavidin and trivalent nodes. FIG. 13G shows a schematic of a hexagon nanostructure formed using streptavidin and single-chain bivalent nodes. FIG. 13H shows a nanohexagon constructed of a combination of streptavidin and single-chain bivalent nodes.

FIGS. 14A through 14C: Electron microscopy of gCA hexagonal lattice nanostructure formation. FIG. 14A shows a schematic illustration of a hexagonal lattice formed through the assembly of trivalent biotinylated nodes and streptavidin. FIG. 14B shows a molecular model of the structure based on a trivalent node construct of the Methanosarcina thermophila 1thj gCA structure to the scale of the electron microscope image shown in FIG. 14C. FIG. 14C shows a uranyl acetate negatively stained region of an electron microscope grid showing the formation of regions of hexagonal nanostructure prepared using streptavidin and a trivalent construct of the Methanosarcina thermophila 1thj gCA.

FIGS. 15A through 15C: Electron microscopy image reconstruction of gCA single chain construct. FIG. 15A shows 60 electron microscope images of isolated molecules of a single-chain node construct of the Methanosarcina thermophile 1thj gCA. FIG. 15B shows a computer-averaged reconstruction of the images based on mathematical correlation and superposition. FIG. 15 C shows the molecular surface computed from Methanosarcina thermophile 1thj gCA engineered structure atomic coordinates.

FIGS. 16A through 16C: Electron microscopy of gCA hexagon nanostructure formation. FIG. 16A shows a schematic illustration of a hexagon nanostructure formed through the assembly of bivalent single-chain biotinylated nodes and streptavidin. FIG. 16B shows a molecular model of the nanohexagon structure based on a bivalent single-chain node construct of the Methanosarcina thermophila 1thj gCA structure to the scale of the electron microscope image shown in FIG. 16C. FIG. 16C shows a negatively stained region of an electron microscope grid with nanohexagons prepared using streptavidin and a bivalent single-chain construct of the Methanosarcina thermophila 1thj gCA.

### DETAILED DESCRIPTION OF THE INVENTION

Carbonic anhydrase enzymes are widely found in nature and catalyze the reversible interconversion of CO₂ and bicarbonate with high efficiency.

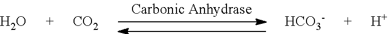

Previous work has investigated the use of carbonic anhydrase (CA) enzymes as catalytic elements in systems designed to scrub CO₂ from closed atmospheric environments and/or industrial exhaust streams (Ge et al. 2002).

In this document, the term “thermostable” can be understood to mean having stability of tertiary and quaternary structure at temperatures of about 50° C. or greater. The term “hyperthermostable” can be understood to mean having stability of tertiary and quaternary structure at temperatures of about 70° C. or greater.

In this document, indication of a protein having “80 percent or greater sequence identity” with the sequence of another protein is to be understood as including, as alternatives, proteins that are required to have a higher percentage of sequence identity with the other protein. For example, alternatives include proteins that have about 80, 85, 90, 95, 98, 99, 99.5, or 99.9 percent or greater sequence identity with the sequence of the other protein. One of skill in the art would understand that given a second amino acid sequence having 80 percent or greater sequence identity to a first amino acid sequence, the three-dimensional protein structure of the second amino acid sequence would be the same or similar to that of the first amino acid sequence. “80 percent or greater sequence identity” can mean that the linear amino acid sequence of a second polypeptide, whether considered as a continuous sequence or as subsections of amino acid sequence of ten or more residues (the order of the subsections with respect to each other being preserved), has identical amino acid residues with a first polypeptide at 80 percent or greater of corresponding sequence positions. For example, a second polypeptide having 20 percent or less of the amino acid residues of a first polypeptide replaced by other amino acid residues would have “80 percent or greater sequence identity”. For example, a second polypeptide having every eleventh residue of a first polypeptide deleted would have “80 percent or greater sequence identity” to the first polypeptide, because each string of ten amino acids of the second polypeptide would be identical to a string of ten amino acids of the first polypeptide—such a second polypeptide would have 10/11=91% sequence identity to the first polypeptide. For example, a second polypeptide having an additional residue inserted after every ten amino acids of a first polypeptide would have “80 percent or greater sequence identity” to the first polypeptide—such a second polypeptide would have 10/11=91% sequence identity to the first polypeptide. For example, this document is to be considered to include those protein sequences herein and having 80 percent or greater sequence identity to the amino acid sequences listed. According to the invention, certain residues can be more important to the structural integrity, symmetry, and reactivity of the proteins, and these must be more highly conserved, while other residues can be modified with less of an effect on the node protein. Generally, proteins that are homologous or have sufficient sequence identity are those without changes that would detract from adequate structural integrity, reactivity, and symmetry.

Standard one-letter and three-letter abbreviations are used for amino acids in this text (unless otherwise indicated).

Protein-based nanotechnology described herein includes the concept of interconnecting multimeric proteins having plane or point group symmetry (“nodes”), with streptavidin or other proteins (“struts”) to form linear interconnections between nodes. The nanostructures can be used for biosensor applications.

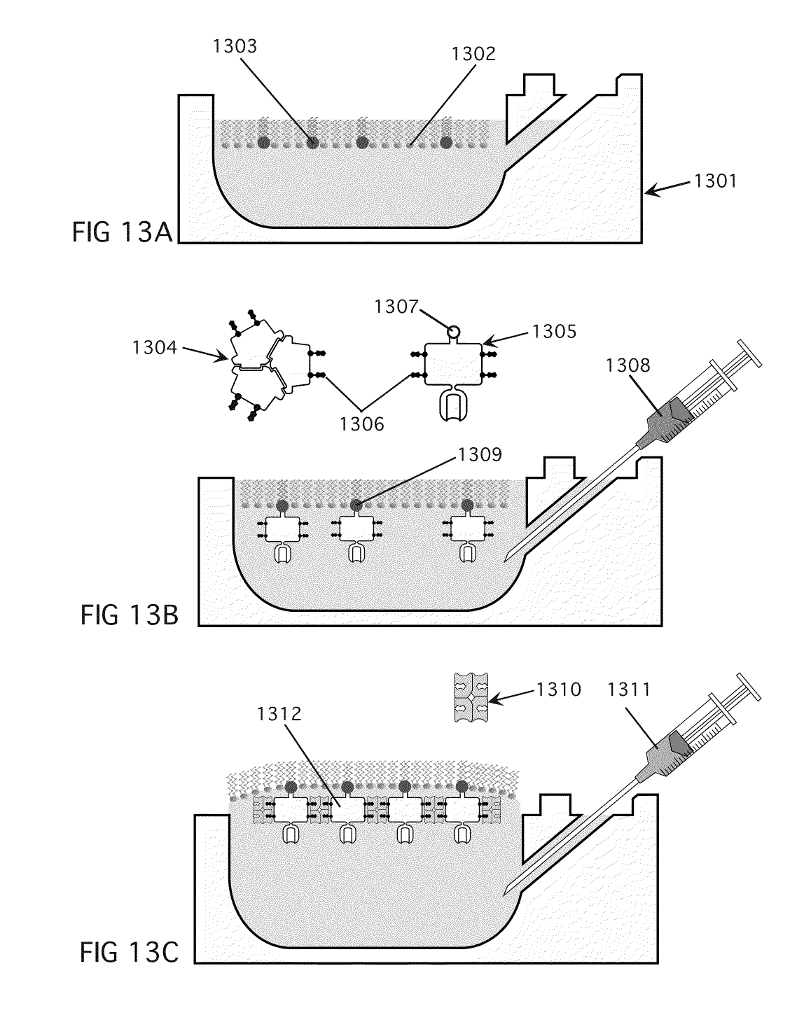

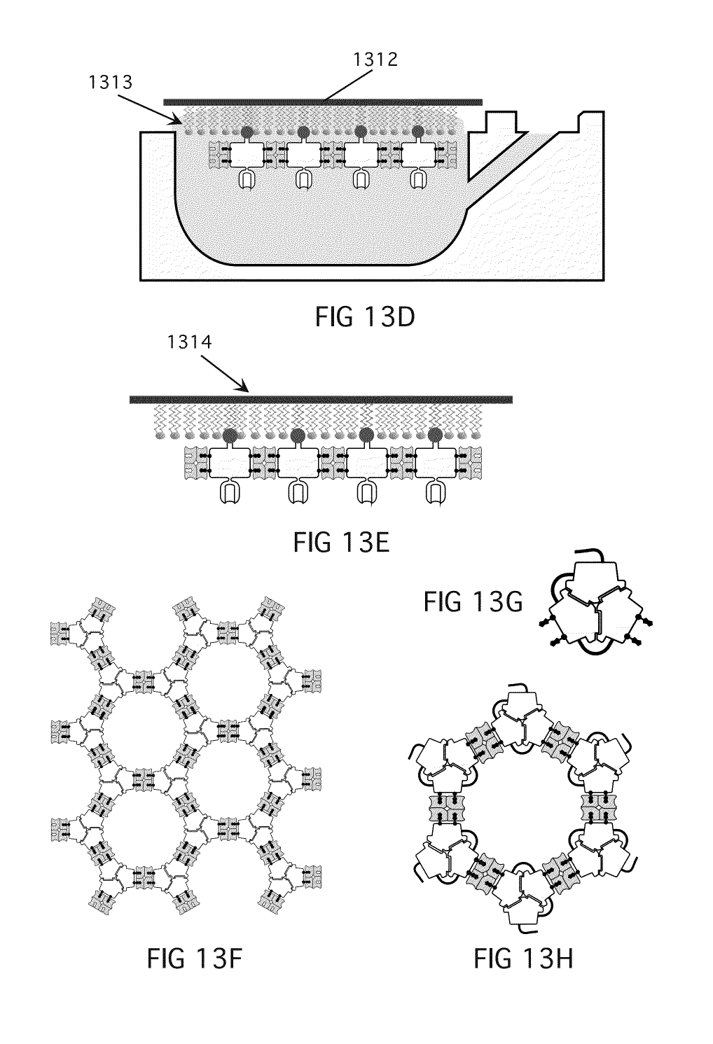

In this description and the associated claims, geometrical and other terms are used to describe structures formed. As a person having ordinary skill in the art will appreciate, the meaning of such geometrical and other terms in the context in which they are used may vary from the idealized definition of the geometrical and other terms. For example, certain structures are referred to as “two dimensional”. In context, as a person of ordinary skill would recognize, the term “two dimensional” encompasses structures with a limited and/or an approximately constant extent in a third dimension, and a much greater extent in the first and second dimensions. For example, a piece of letter-sized writing paper can be described as “two dimensional”. For example, the protein nanostructure illustrated in FIG. 13F can be described as “two dimensional”. The terms “plane” and “planar” have a similar meaning here.

A person having ordinary skill in the art would understand a tessellation, tiling, or lattice as a two-dimensional structure in which a cell or tile or unit which remains substantially constant is adjacently repeated in two dimensions. There can be some variation in the cells or tiles for the structure formed to still be considered a tessellation or tiling. A tessellation, tiling, or lattice can be finite in extent. The extent of a tessellation, tiling, or lattice can be defined as a finite number of units. For example, a tessellation, tiling, or lattice according to the invention may extend 2, 4, 10, 20, 40, 100, 500, 1000, or more units, or an intermediate amount. For example, a tessellation can be a triangular tessellation (having cells resembling triangles), a square tessellation (having cells resembling squares or rectangles), or a hexagonal tessellation (having cells resembling hexagons).

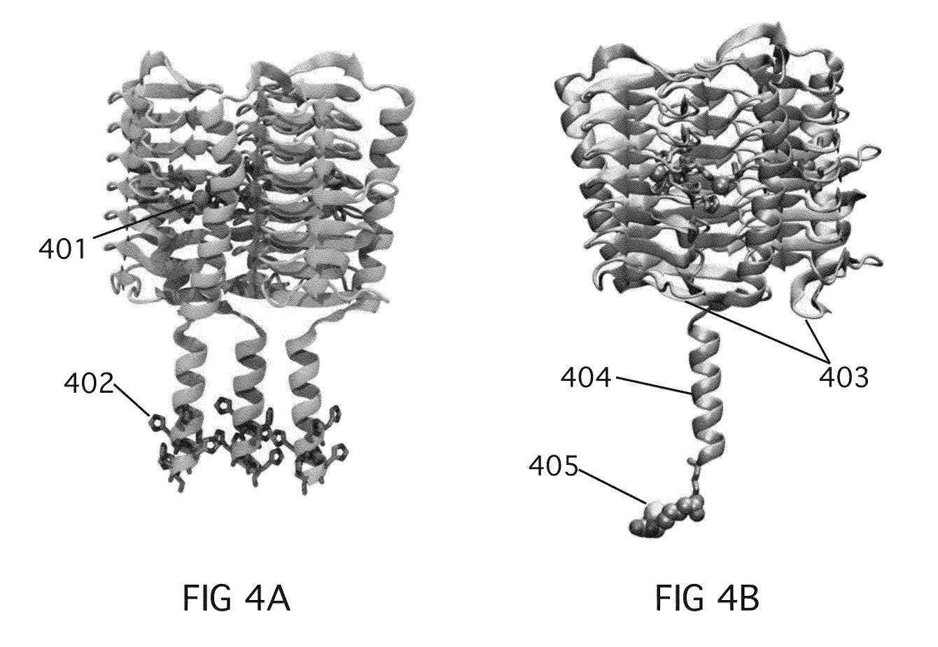

A C3 symmetric object can be an object that appears substantially identical when rotated in increments of 120 degrees about an axis. The object can still be described as C3 symmetric if there is some variation in appearance when rotated in an increment of 120 degrees. For example, a protein trimer having 3 subunits linked together as a single polypeptide chain can be described as C3 symmetric, even though the first and third subunits are each linked through amino acid residues only to the second subunit, whereas the second subunit is linked through amino acid residues to both the first and the third subunits. In some contexts, such a protein trimer having 3 subunits linked together as a single polypeptide chain can be described as having reduced symmetry (as compared to the native protein trimer formed of three (3) separate, identical subunits). For example, the single-chain trimer node illustrated in FIG. 4A can be described as being C3 symmetric or can be described as having reduced symmetry.

A trimer node can be a C3 symmetric protein trimer. A node can connect or bind to one strut or connect or bind two or three struts together and orient them in a predetermined geometry by the node binding to the strut(s). A strut can be protein, such as streptavidin, that functions as a linear connector. For example, a first trimer node can bind to one end of a strut, and a second trimer node can bind to the opposite end of the strut. The strut can thereby fix the spacing and orientation of the two timer nodes with respect to each other. For example, FIG. 4C illustrates trimer nodes connected together by struts.

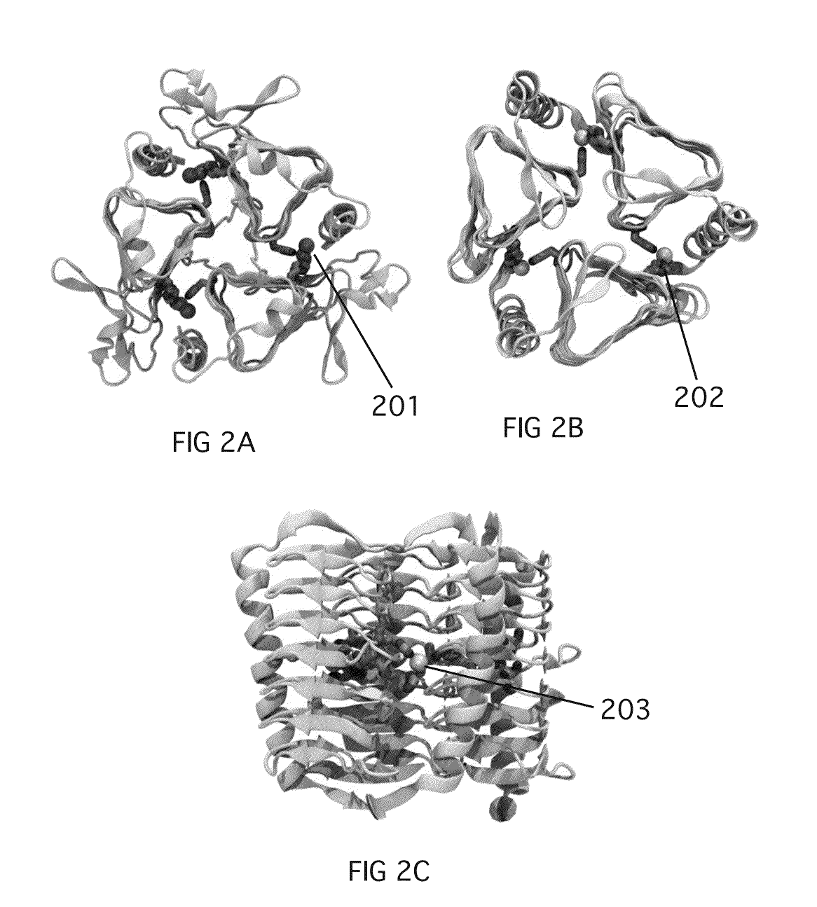

“Valency” can refer to the number of other objects which a given object can bind. For example, a trivalent trimer node, such as illustrated in FIG. 2A, can bind three streptavidin struts. For example, a bivalent timer node, such as illustrated in FIG. 4A, can bind two streptavidin struts. For example, a monovalent trimer node, such as illustrated in FIG. 4B, can bind one streptavidin strut.

The description of embodiments and methods of the invention described herein and the meaning of terms used is to be informed by the Figures in the drawings which form part of this specification. A person having ordinary skill in the art can understand the terms and their use in the context of the text in which such terms are used and the Figures that complement the text.

### CO₂ Scrubbing Apparatus:

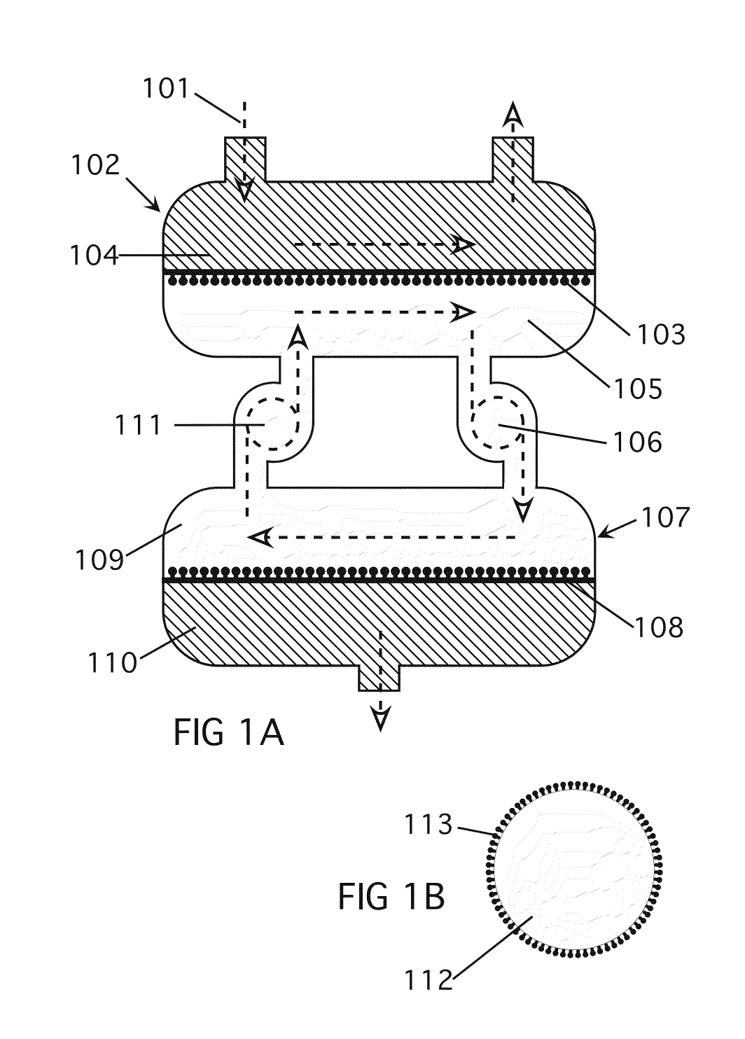

In one application, the first separation stage of a CO₂ scrubbing apparatus incorporates an asymmetric, semipermeable membrane having an immobilized enzyme exposed to a flowing fluid phase on one side, and the gas stream containing CO₂ on the other side. During operation, CO₂ from the gas stream diffuses across the semipermeable membrane into the liquid phase where it is converted into bicarbonate through the action of the immobilized CA enzyme. Removal of the bicarbonate from the liquid transfer phase can take place by reversing the process, using a second CA-substituted membrane system to convert bicarbonate back into CO₂, or by other means. FIG. 1A shows a schematic of such a system that transfers CO₂ from a closed environment (e.g. a spaceship or space suit) to an open environment (a space atmosphere outside the space ship or space suit). In this apparatus, the interconversion of CO₂ and bicarbonate is catalyzed by carbonic anhydrase enzyme molecules that are immobilized on an asymmetric membrane surface. In such an apparatus, a gas stream 101 containing CO₂ is admitted to a chamber 102 that is divided by an asymmetric semipermeable membrane 103. The semipermeable membrane 103 is exposed to the gas stream environment 104 on one side of the semipermeable membrane, and to a liquid carrier environment 105 on the other side. Carbonic anhydrase enzyme molecules immobilized on the liquid-exposed side of the semipermeable membrane 103 catalyze the conversion of CO₂ diffusing across the membrane into bicarbonate anion that dissolves in the liquid phase contained in the volume 105. A pump 106 moves the bicarbonate-enriched liquid into a second chamber 107 that is divided by a second asymmetric semipermeable membrane 108. The membrane, incorporating surface-bound carbonic anhydrase enzyme molecules, catalyzes the conversion of bicarbonate anion present in the liquid chamber 109 into CO₂, which diffuses across the membrane 108 into the gas-containing chamber 110 where the gas can exhaust or otherwise be removed. A second pump 111 optionally assists in recirculating the bicarbonate transfer fluid between chambers 102 and 107.

An alternative application shown in FIG. 1B immobilizes engineered forms of carbonic anhydrase enzyme molecules on the surface of resin particles or other bead materials that are suitable for packing in beds or columns incorporated in CO₂ scrubbing apparatus.

### gCA Enzymes:

There are numerous forms of CA enzyme present in nature. The present invention describes engineered forms of thermostable gamma-CA (gCA) enzymes that offer key advantages in production and use in CO₂ scrubbing applications. The engineered enzymes are designed to meet several requirements that enable practical CO₂ scrubbing applications. These include 1) low enzyme production cost and ease of isolation, 2) high catalytic turnover rate, 3) useful lifetime the integrated apparatus, and 4) ability to be reversibly immobilized on the reactor surface to allow apparatus recharging. As detailed below, the trimeric gCA enzymes incorporate structural features that allow them to be modified to allow controlled and reversible immobilization to solid surfaces such as presented in the scrubber applications outlined in FIGS. 1A through 1B.

The inventions described utilize a combination of computational modeling and recombinant DNA technology to design and produce modified gCA enzymes having the required functional characteristics. The engineered enzyme constructs are designed to allow controlled, oriented immobilization of the gCA enzymes with offsets from an immobilization surface designed to optimize reaction efficiency. Constructs described incorporate either one or three immobilization sites per enzyme trimer, and employ different forms of immobilization chemistry. In addition to providing optimal immobilization geometry to maximize enzyme activity, the immobilization sequences are designed to offer low leakage from the immobilization surface, but also to allow the formation of reversible linkages, so allowing the CO₂ scrubbing apparatus to be “recharged” when the requirement arises to replace the active catalyst owing to degradation of activity under use conditions in the field.

### gCA Enzyme Structural Properties:

FIGS. 2A through 2C outline the 3D structural properties of two gCA enzymes known from X-ray crystallography. These include the gCAs isolated from the thermophilic microorganism Methanosarcina thermophila (www.rcsb.org pdb code 1thj, Kisker et al. 1996) and from the extreme thermophile Pyrococcus horikoshii OT3 (www.rcsb.org pdb code 1v3w, Jeyakanthan et al. 2008). FIG. 2A shows a projection down the C3 symmetry axis of the trimeric 1thj gCA. The label 201 designates one of the catalytic zinc atoms of the trimer that is ligated to 3 histidine residues. FIG. 2B shows a projection down the C3 symmetry axis of the 1v3w trimeric gCA. The label 202 designates one of the catalytic zinc atoms of the trimer that is ligated to 3 histidine residues. FIG. 2C shows a side view of the 1v3w gCA trimer. The label 203 designates one of the catalytic zinc atoms of the trimer.

The 1thj and 1v3w native proteins are trimers with each subunit organized as a left-handed beta-coil that rises from the “base” of the molecule to the “top”, where the polypeptide chain reverses direction and descends to the base in an alpha-helical conformation. The active sites of the gCA enzymes incorporate a catalytic zinc atom coordinated by three histidine imidazole side chains situated at the interface of adjacent subunits. The most direct access to the three active sites in the trimeric structures occurs through channels on the top and side of the structures. Studies of the 1thj-gCA from Methanosarcina thermophila demonstrate a thermal stability of 55 degrees C. (Kisker et al. 1996) and a turnover rate that depends on a variety of factors, including the nature of bound metal ions and operating pH range, with observed turnover rates of up to 2×10⁵ sec⁻¹ for proteins grown under conditions that insure optimal catalytic Zn incorporation (Zimmerman et al. 2010). Other studies have shown that the turnover of the Zn-ligated enzyme can be further enhanced by up to 40% by exchanging the catalytic Zn with Co (Alber et al. 1999). Less is known about the specific catalytic properties of the Pyrococcus horikoshii 1v3w gCA, although it is thermally stable to 90 degrees C. (Jeyakanthan et al. 2008). An important factor evidently contributing to the enhanced thermal stability of 1v3wgCA is the coordination of multiple Ca⁺⁺ ions by protein side chain carboxyl groups. In the present invention, we describe engineered gCAs constructs based on both the thermophile 1thj and hyperthermophile 1v3w proteins. In particular, we note that the lower overall molecular weight and higher thermal stability of the Pyrococcus horikoshii 1v3wgCA will offer advantages in production and process stability relative to the less-thermostable Methanosarcina thermophila gCA enzymes. In addition, the engineered modifications proposed are applicable to several additional gCAs derived from extreme thermophiles that have sequence homology and structural homology with the 1v3w and/or 1thj proteins.

Both optimization of production and maintenance of enzyme catalytic capacity are greatly facilitated by using CA enzymes derived from thermophilic organisms. Such proteins have enhanced thermal and chemical stability that makes them easy to isolate following expression in E. coli. fermentation systems, generally facilitates steps required in device fabrication, and provides functional longevity in the end use CO₂ scrubbing apparatus.

As noted above, important factors limiting the effectiveness of CO₂ scrubbing using immobilized CA enzymes include loss of enzyme activity owing both to lack of geometrical control over the CA enzyme immobilization process, and chemical damage to the enzymes incurred through the harsh chemical conditions required for immobilization. The novel aspects of the present constructs include engineered structural features that 1) immobilize the enzyme to allow maximal catalytic activity when bound on support substrates like membranes and beads, 2) incorporate specific immobilization sequences that allow high affinity immobilization to, and low leakage from, the process substrate surface without requiring harsh chemical conditions, and 3) also form reversible interactions, so that the active substrate surface can be stripped of immobilized enzyme and the scrubbing apparatus recharged with new enzyme in the field.

The present invention describes alternative approaches to achieving the objectives outlined above that include alternative immobilization chemistry and engineered forms of both trimeric and single-chain engineered constructs of the gCA enzymes.

### Trimeric gCA Constructs:

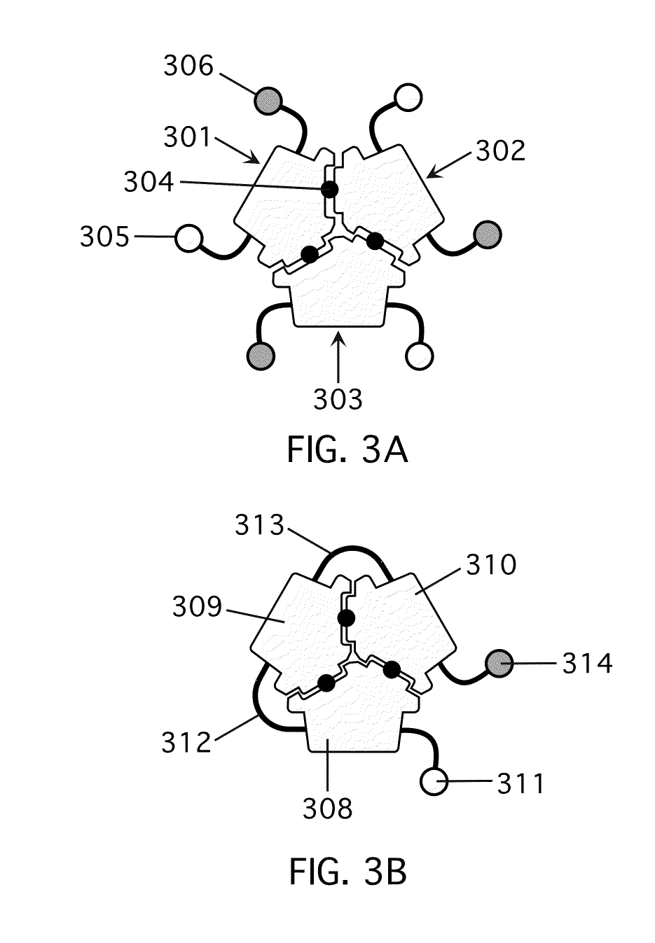

FIG. 3A shows a schematic illustration of a trimeric, engineered, gCA enzyme construct. As shown in FIG. 3A, the symmetric trimer composed of identical subunits 301, 302, and 303. An active site zinc atom 304 is located at each subunit interface. Each subunit sequence can be modified through addition of an immobilization sequence at either the amino terminus 305 or carboxy terminus 306 of the subunit polypeptide chain.

FIG. 4A shows a backbone side view of an engineered form of the trimeric 1v3w gCA from Pyrococcus horikoshii. The active site zinc of one subunit is shown as 401. The polypeptide chain C-terminus of each subunit has been extended with a poly-His terminal sequence 402 that enables binding the trimer to a Ni-NTA functionalized surface. Although both N and C terminus extensions are geometrically possible, constructs with C-terminus extensions are illustrated and have already demonstrated excellent levels of expression (See Examples below).

Engineering attachment of terminal sequences to one or both of the gCA polypeptide chain termini facilitates a number of means of reversible surface immobilization.

### Ni-NTA Surface Immobilization:

For example, poly-Histidine and related sequences are known to form strong interactions with Ni-NTA (nickel-trinitrilo acetic acid) functionalized surfaces. A number of substrate surface materials may be functionalized with Ni-NTA groups using known methods and chemical reagents. Owing to the multivalent interaction made between each timer and a highly functionalized NiNTA surface, enzyme binding affinity to the membrane is anticipated to approximate a Kd≦10⁻¹³ M. Nevertheless, the poly-His-NTA interaction is reversible at slightly acidic pH and/or in the presence of imidazole, allowing the system to be efficiently recycled.

### Gold Surface Immobilization:

Alternative constructs can be designed to allow immobilization through N and C polypeptide terminal sequences incorporating cysteine-containing sequences (Sasaki et al. 1997). Such sequences have a high affinity for gold surfaces. Proteins bound to surfaces through gold-sulfur linkages may be removed through the use of strong oxidizing agents.

### Amine Reactive Surface Immobilization:

Alternative immobilization linkages can be formed by reacting either the N-terminal amino group of the polypeptide chains or the epsilon amino groups of lysine residues on the protein surface to amine reactive immobilization reagents. Examples of amino immobilization chemistry on e.g. gold surfaces include the use of the reagent dithiobis(succinimidylpropionate) which is a bifunctional S—S linked reagent with an amine-reactive N-hydroxysuccinimide (NHS) ester at each end. The reagent is strongly chemisorbed on gold surfaces leaving the NHS groups free to react with protein amine groups. Owing to the plurality of lysine groups usually found on protein surfaces, the immobilization linkages formed will generally be nonspecific, but can be made specific and lead to controlled terminal immobilization if lysine residues present in the sequence are mutated to arginine or other compatible amino acid residues that lack a side chain group that is able to react with the immobilization reagent. In this case only the amino terminal amine of the protein will be able to react specifically with the NHS groups (Katz, E Y 1990). As is the case for protein immobilized through cysteine side chain interactions, proteins bound to surfaces through gold-sulfur linkages may be removed through the use of strong oxidizing agents.

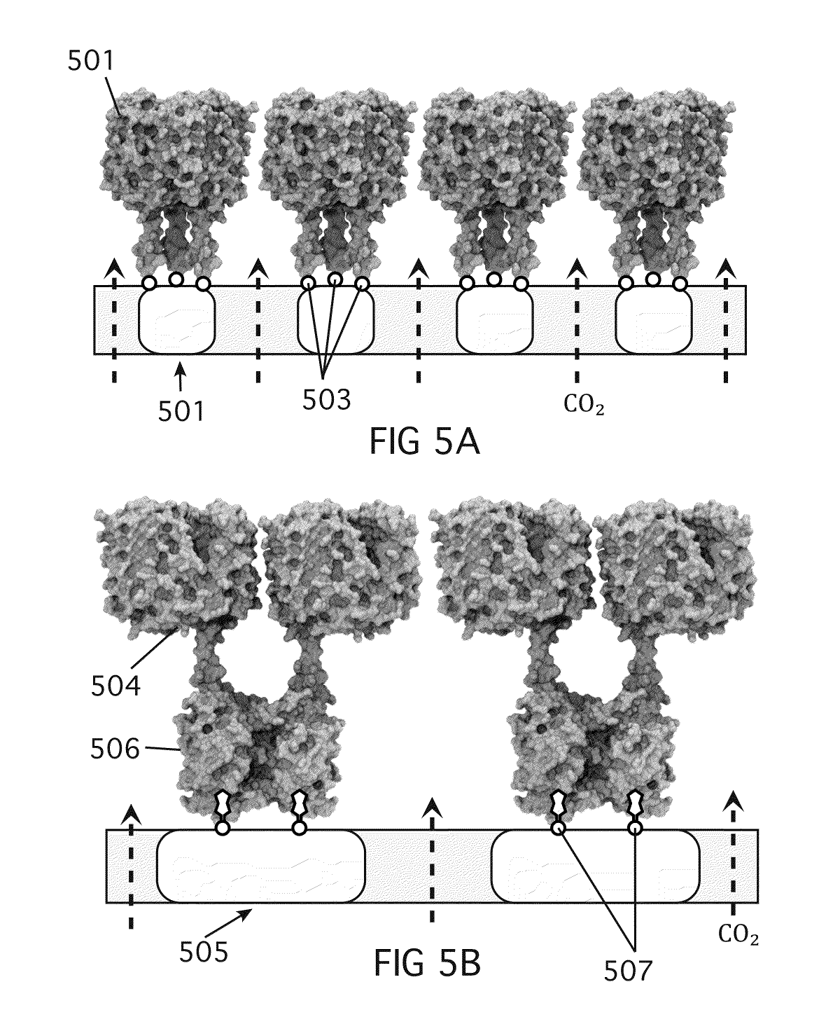

FIG. 5A is a schematic illustration of the engineered 1v3w trimeric gGAs immobilized on the asymmetric membrane surface of a CO₂ scrubbing apparatus. FIG. 5A shows a molecular model of the 1v3w gCA extended-terminus timer 501 bound to a porous membrane substrate 502. Each enzyme trimer is bound to the membrane through 3 chemical linkages 503 formed between the membrane and the protein timer.

### Single-Chain gCA Constructs

Alternative constructs may be generated that incorporate the three subunit chains present in the native enzyme into a single-continuous polypeptide chain. FIG. 3B shows a schematic that outlines the structure of single-chain constructs. As shown in FIG. 3B, in the single-chain construct individual subunit chains 308, 309, 310 have been linked into a single polypeptide chain with linkers 312 and 313. The single-chain structure can be additionally modified through incorporation of an immobilization sequence at either the amino terminus 311 or carboxy terminus 314 of the continuous polypeptide chain.

As noted above (FIGS. 2A through 2C) both the N and C terminii of the monomer subunit polypeptide chains are situated at the “bottom” of the trimeric enzyme molecule. Sequences can be appended to either terminus of the “core” enzyme structure to achieve oriented immobilization. FIG. 4B shows a backbone side view of an engineered form of 1v3w gCA where the trimer has been engineered as a single-chain construct through the introduction of two subunit linkers 403. The C-terminus helix 404 of the single-chain construct has been extended with a substrate sequence that allows the specific enzymatic addition of a covalently bound biotin group 405. Analogous structures exist for the 1thj gCA enzyme.

As outlined in FIG. 4B, owing to the geometry of the 1thj-gCA and 1v3w-gCA proteins, where the N and C termini of the polypeptide chains of adjacent timer subunits are closely situated at the “base” of the trimer, the subunits can be interconnected to form a single polypeptide chain through the introduction of short linking polypeptide loops Immobilization of single chain constructs can employ the either the Ni-NTA surface, gold surface, or amine functionalized surface modes of immobilization as outlined above for immobilization of the engineered trimeric structures.

### Streptavidin Surface Immobilization:

An alternative mode of immobilization, suited particularly to single chain constructs, involves specific biotinylation of the single chain nodes. By incorporating a specific sequence allowing enzymatic biotinylation (e.g. LERAPGGLNDIFEAQKIEWHE (SEQ ID NO: 2)) into the terminal sequence of a single chain construct and, by expressing the engineered protein in an E. coli expression system that also includes the associated enzymatic components (Barat & Wu 2007, Chapman-Smith & Cronan, 1999), it is possible to isolate the engineered, terminally biotinylated proteins (FIG. 4B) directly from the expression system hydrolysate. The sequences introduced represent a substrate for E. coli biotin ligase that covalently attaches biotin to the protein, a post-translational modification that is exceptionally specific and widely used to purify proteins expressed in E. coli (Kay et al. 2009).

Surface immobilization of the biotinylated single-chain gCA enzyme constructs will be facilitated by crosslinking to the substrate with streptavidin. Streptavidin is a tetrameric protein of ˜60,000 MW that binds 4 biotin molecules at binding sites roughly configured as the legs of an “H” (Weber et al. 1989). The affinity of streptavidin for biotin is approximately Kd≦10⁻¹⁴M, which makes the interaction practically irreversible and has led to the wide utilization of the biotin-streptavidin interaction in biotechnology applications. In addition, biotin-complexed-streptavidin is itself stable, with a thermal denaturation temperature >80 degrees C. (Weber et al. 1989, 1992, 1994). Streptavidin is structurally homologous to the tetrameric biotin binding protein avidin (Repo et al. 2006). Consequently, forms of avidin can be used alternatively to streptavidin in the nanostructure constructs and immobilization applications described here.

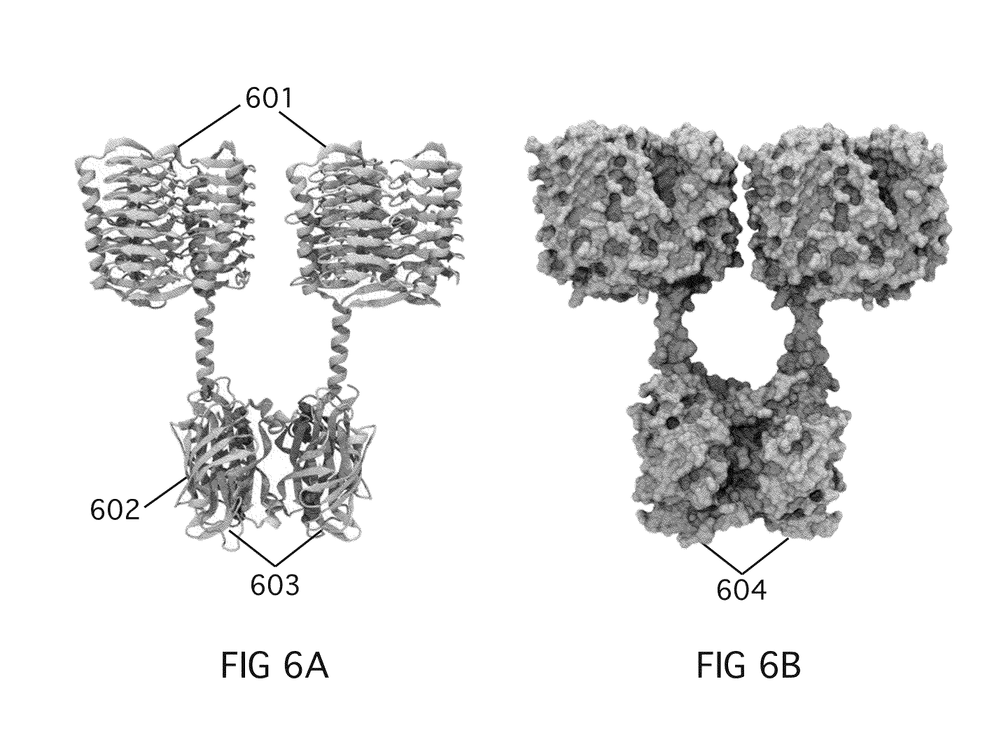

FIGS. 6A through 6B outline the molecular structure of the streptavidin complex with two biotinylated gCA single-constructs bound. FIG. 6A shows a ribbon model of two single-chain biotin-linked gCAs 601 (also FIG. 4B) bound to a surface-immobilized streptavidin tetramer 602. The streptavidin is immobilized by two surface bound biotin groups that can bind a pair of biotin-binding sites 603 on the streptavidin tetramer. FIG. 6B shows a molecular surface representation of the complex showing the position of the surface immobilization sites 604. FIG. 5B shows the assembly immobilized on membrane surface.

Owing to the pairwise orientation of the binding sites in streptavidin, the gCA surface immobilization process will first immobilize streptavidin on the a biotinylated substrate surface, which as a geometrical consequence of the situation of the biotin binding sites on the streptavidin tetramer, will leave half of the biotin binding sites on each tetramer open. Subsequent addition of the biotinylated constructs will immobilize the biotinylated gCA single-chain constructs to produce the assembly shown in FIG. 5B. FIG. 5B shows a molecular model of the 1v3w biotinylated single-chain gCA 504 bound to a porous membrane substrate 505 through an intermediate streptavidin tetramer 506. The structure is formed by first immobilizing streptavidin to surface biotinylation sites 507. Both the immobilization schemes shown in FIGS. 5A and 5B tile 2D surfaces with enzyme timers on ˜5 nM lattice centers.

Despite the high affinity of the biotin streptavidin interaction, recent work reports the reversibility of the interaction at 70 deg C. using deionized water (Holmberg 2005), providing a particularly simple means for apparatus regeneration in the field.

Control of gCA enzyme immobilization to provide a reversible system with high turnover and low leakage defines a key performance objective of the engineered enzymes in integrated systems for CO₂ scrubbing.

### Streptavidin-Linked gCA Nanoassemblies

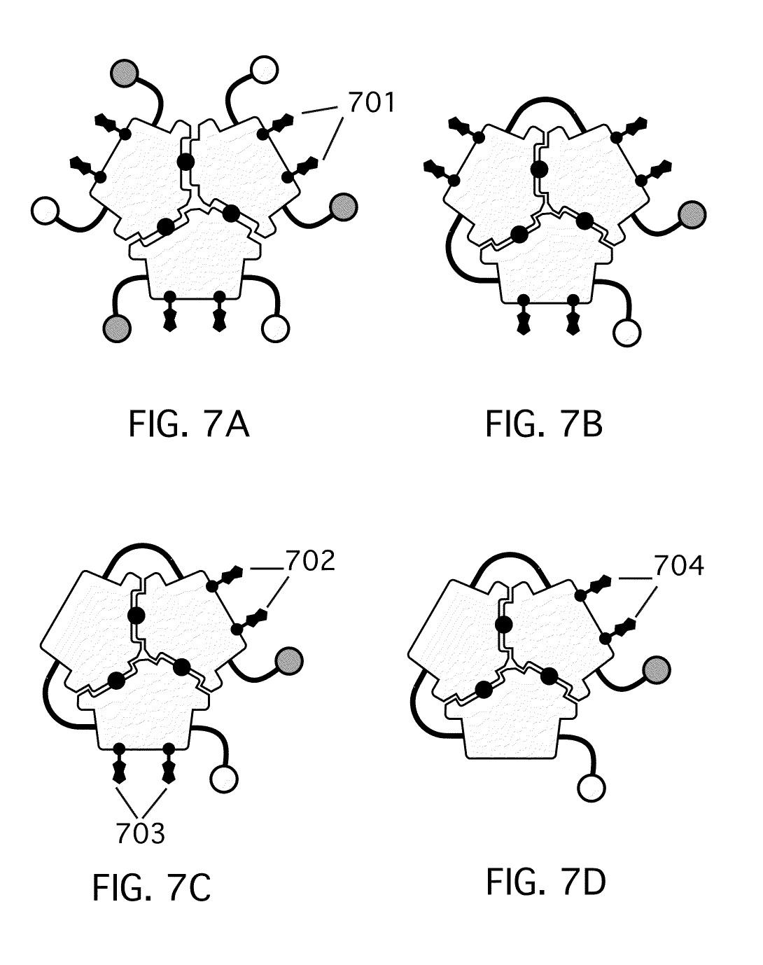

Enhanced utility of immobilized gCA constructs that reduce unwanted dissociation of enzyme from reactor surfaces can be achieved through the formation of nanoassemblies where individual enzyme trimers or single-chain constructs are interconnected, so that connected enzyme complexes make multiple linked interactions with the reactor apparatus substrate. The multiplicity of interactions and interconnectivity of the interactions thus formed make the nanoassembly highly resistant to dissociation from the reactor substrate surface. Engineered forms of gCA trimer can be designed where two cysteine substitutions are introduced into the polypeptide sequence of each subunit, providing specific chemical sites that can be biotinylated using one of several cysteine-reactive biotinylation reagents. The binding sites are designed using computer modeling methods (See Examples below) so that the biotinylation sites are complementary to pairs of biotin binding sites on the tetrameric biotin-binding protein streptavidin. FIG. 7A shows a schematic of a symmetric gCA trimer composed of identical subunits where each subunit has been modified to incorporate 2 covalently bound biotin groups 701. The trimer can consequently form a trivalent interaction with three streptavidin tetramers.

### Trigonal Scaffold:

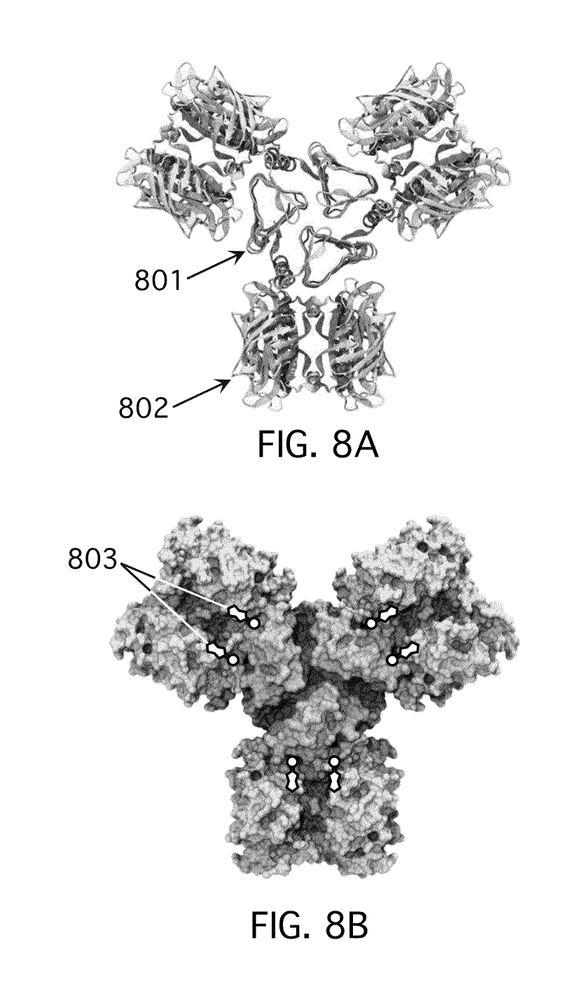

FIG. 8A shows a backbone ribbon representation of the 1v3w gCA trimer 801, where each subunit has been modified to incorporate 2 covalently bound biotin groups that allow binding to a streptavidin tetramer 802. FIG. 8B shows a molecular surface representation of the complex of FIG. 8A, indicating the projected positions of the biotin residues 803 that interconnect the central node with the peripherally bound streptavidin tetramers. The pre-assembled trigonal “scaffold” of FIGS. 8A through 8B is a key component in the formation of numerous nanoassemblies described below.

### Trivalent, Bivalent, and Monovalent Single Chain Constructs:

As outlined above (FIGS. 3A through 3B) the individual subunits of the gCA trimer structure can be interconnected form a continuous polypeptide chain. FIG. 7B schematically shows a single-chain trivalent gCA construct able to form interactions with 3 streptavidin tetramers. However, formation of single-chain gCA constructs also allows precise control over which enzyme trimer subunits can be modified by cysteine introduction to allow biotinylation and subsequent formation of streptavidin complexes. For example, FIG. 7C shows a single-chain construct where two pairs of biotinylation sites, 702 and 703, have been incorporated in the single-chain construct to produce a bivalent node able to bind two streptavidin tetramers. FIG. 7D shows a single-chain construct where a single pair of biotinylation sites, 704, have been incorporated in the single-chain construct to produce a monovalent node able to bind a single streptavidin tetramer. Connection of the trimer subunits into a single continuous polypeptide chain allows the C3 symmetry of the timer to be broken, producing for example, single-chain constructs can be made that form bivalent (FIG. 7C) and monovalent (FIG. 7D) interactions with streptavidin.

### Hexagonal Nanostructures:

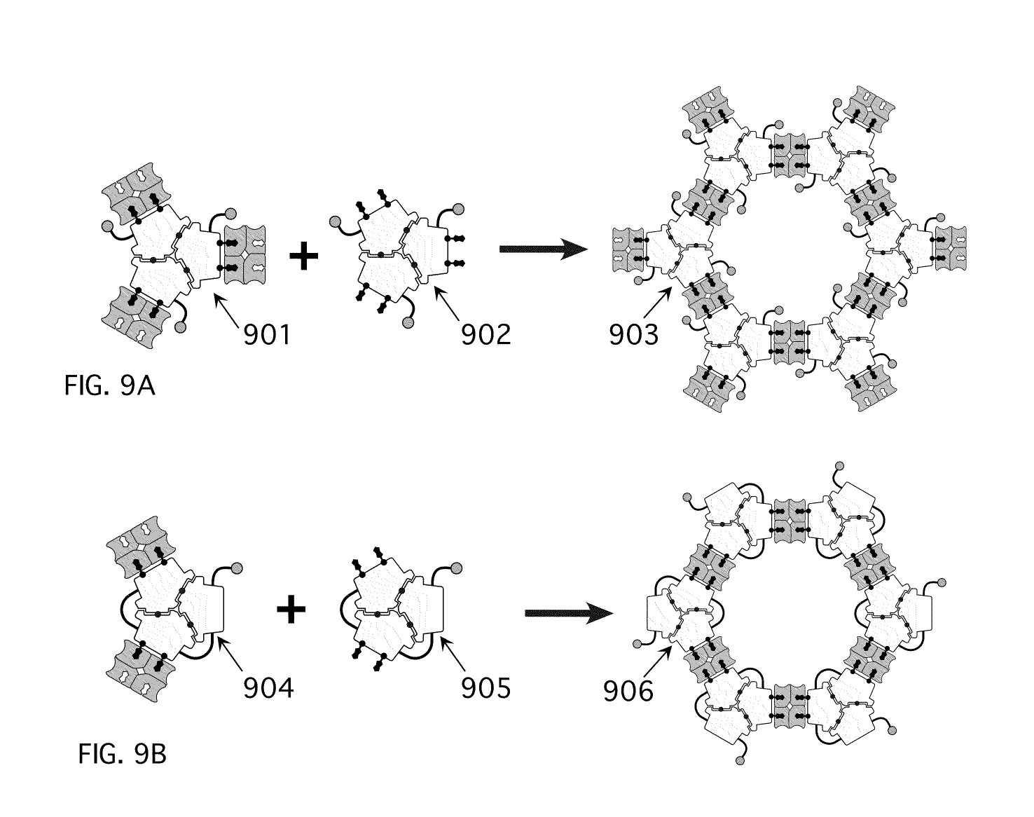

FIGS. 9A through 9B illustrate the formation of hexagonal surface structures formed on 2D surfaces. FIG. 9A outlines an efficient process of gCA hexagonal lattice nanostructure assembly. A trivalent trimeric gCA construct pre-saturated with three streptavidin tetramers to form the complex 901 is combined with free trimeric gCA 902 to form the hexagonal lattice 903. FIG. 9B outlines an efficient process of gCA hexagon nanostructure assembly. A bivalent single-chain gCA construct pre-saturated with two streptavidin tetramers to form the complex 904 is combined with free bivalent single chain gCA construct 905 to form the closed hexagon 906. Hexagonal lattice assembly using a combination of preassembled trigonal scaffold structures (FIGS. 8A through 8B) and individual trivalent nodes reduces the overall molecularity of the assembly process, which improves the assembly efficiency and quality. FIG. 9B illustrates that hexagon nanostructures can be formed using a combination of bivalent single-chain nodes and streptavidin. Again, pre-assembly of streptavidin-bivalent node complexes reduces the molecularity and improves efficiency of the assembly process.

### Trigonal Nanostructures:

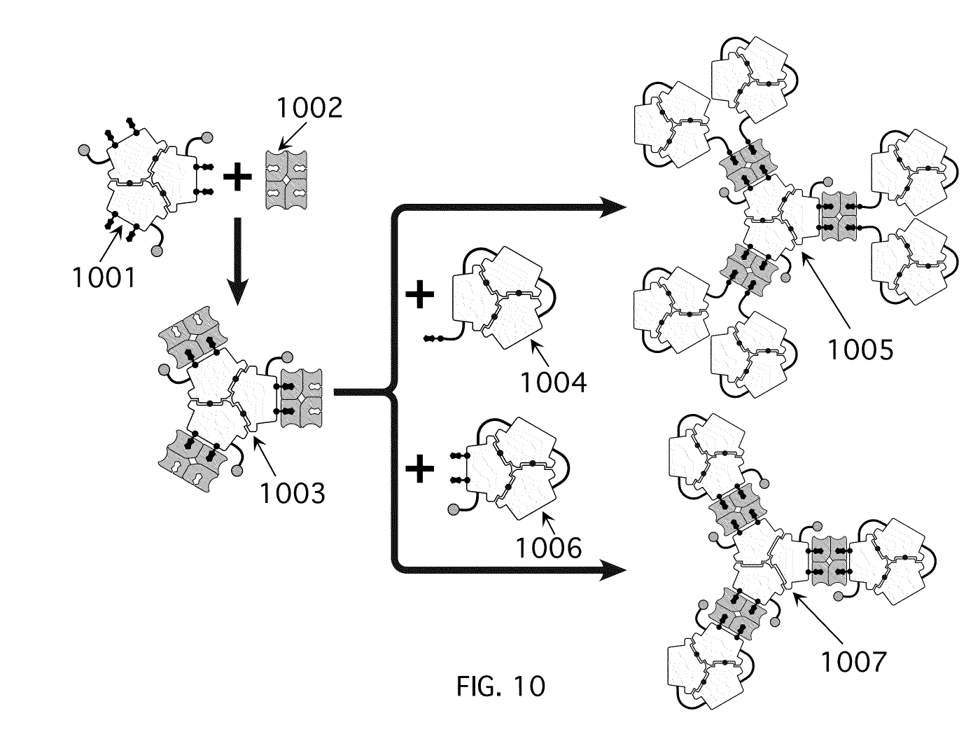

The preassembled trigonal scaffold of FIGS. 8A through 8B can be also used to create different trigonal or “propeller-shaped” gCA nanostructures. FIG. 10 illustrates the formation of trigonal nanostructures can be can be formed using a combination of trivalent nodes (FIG. 7A), monovalent nodes (FIG. 7C) and streptavidin. To assemble the nanostructures, the trivalent gCA node 1001 is initially combined with 3 streptavidin tetramers 1002 to form the trigonal scaffold 1003. The trigonal scaffold 1003 can be combined with the terminally biotinylated single-chain gCA construct 1004 to form the trigonal gCA nanoassembly 1005. Alternately, the trigonal scaffold 1003 can be combined with the monovalent, di-biotinylated single-chain gCA construct 1006 to form the trigonal gCA nanoassembly 1007.

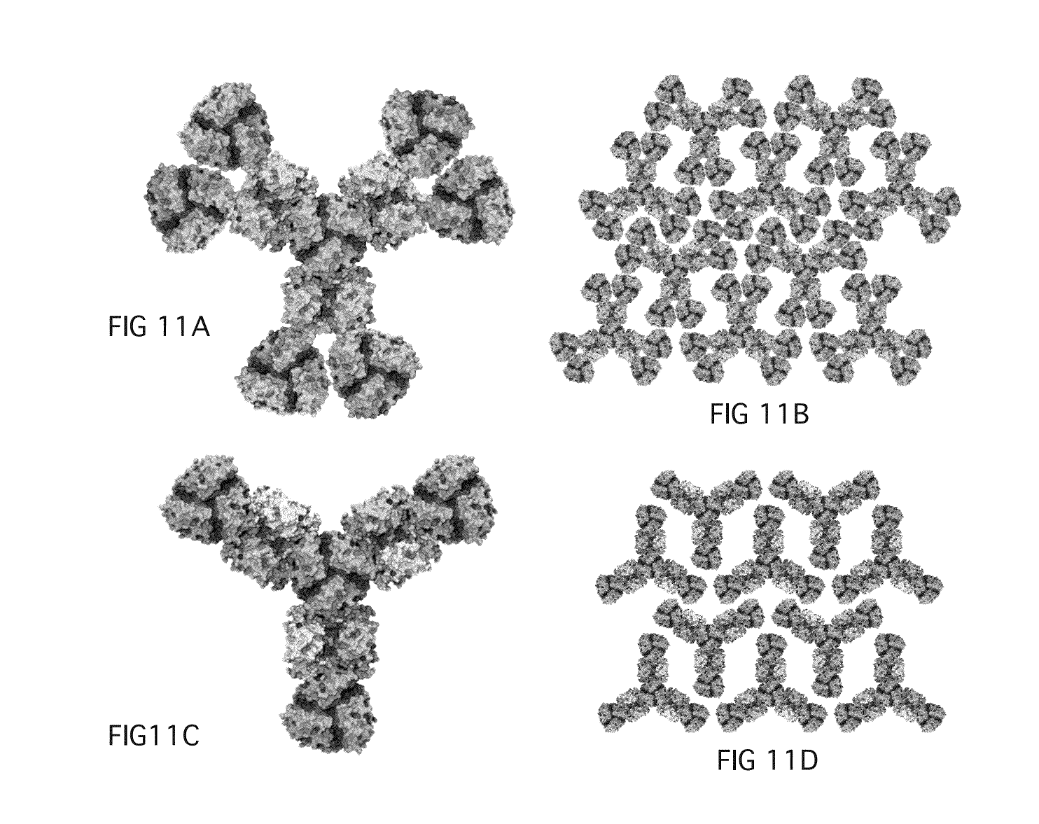

A key advantage of trigonal constructs is that they can be assembled through a sequential process where each step to form a pre-assembly can be driven by mass action. This aids in the preparation of highly purified material. Nanostructures based on the trigonal scaffold assembly platform have the additional useful property that they can continuously tile a surface to provide a high density of enzyme catalytic sites. For example, FIG. 11A shows a molecular model of the trigonal nanoassembly schematically illustrated in FIG. 10-1005 based on the 1v3w gCA molecular structure. FIG. 11B illustrates that the nanoassembly of FIG. 11A can efficiently tie a 2D surface. FIG. 11C shows a molecular model of the trigonal nanoassembly schematically illustrated in FIG. 10-1007 based on the 1v3w gCA molecular structure. FIG. 11D illustrates that the nanoassembly of FIG. 11C can efficiently tile a 2D surface at high density.

In summary, formation of 2D gCA structures with streptavidin crosslinks can require careful control of assembly conditions owing to the essential irreversibility of the streptavidin:biotin binding interaction (Kd˜10⁻¹⁴M). However, by forming structures using a pre-assembled trigonal scaffold (FIGS. 8A through 8B) as outlined above, a variety of nanostructures (FIGS. 9A through 9B and 10) can be produced. Most notably, trigonal nanoassemblies can be assembled in a step-wise fashion where each step can be driven to completion through mass action (FIG. 10), thus greatly enhancing assembly efficiency and final quality. In addition, as shown in FIGS. 11A through 11D, trigonal nanoassemblies can also form closely tiled interactions on surfaces, and so provide a high density of gCA catalytic sites in CO₂ scrubbing applications.

### EXAMPLES

### Engineered Protein Design

Engineered gCA constructs were designed using a combination of heuristic protein modeling tools (Finzel et al. 1990, Guex et al. 1999), computational energy methods (Case et al. 2005), and custom computer codes. For nodes designed for streptavidin-linked nanostructure formation, specific amino acid substitution sites on the surface of the node proteins for mutation to cysteine were determined using a combination of geometrical methods and constrained intermolecular docking protocols. Sites for conversion to cysteine residues were identified using these methods that when derivatized with thiol-reactive biotinylation reagents, would situate two covalently bound biotin groups in positions that accurately corresponded to two, approximately collinear biotin binding sites on the streptavidin tetramer. Terminal sequences, inserted functional domains, and single-chain inter-subunit linkages were geometrically determined using fragment superposition modeling tools (Finzel et al. 1990, Guex et. al 1999), and evaluated for geometrical sequence compatibility and proteolysis resistance. The design process produces both anticipated 3-dimensional structures for the engineered constructs and a corresponding linear amino acid sequence. Table 1 lists sequences for several engineered gCA constructs that incorporate core amino acid sequence elements from the Methanosarcina thermophila (www.rcsb.org pdb code 1thj) Pyrococcus horikoshii OT3 (www.rcsb.org pdb code 1v3w) gCA enzymes. Table 2 lists sequences additional thermostable gCA enzymes that may be used interchangeably with the 1thj and 1v3w core structures to form engineered constructs with similar molecular structure and properties. Amino acid sequences in Tables 1 and 2 are provided using the standard one letter representation for each amino acid. In the examples of the synthetic gene and expression vector sequences shown below, the vector sequence is in lower case with the promoter underlined and the ribosome binding site in italics, and the open reading frame is in upper case with the initiating Methionine and Stop codons in bold.

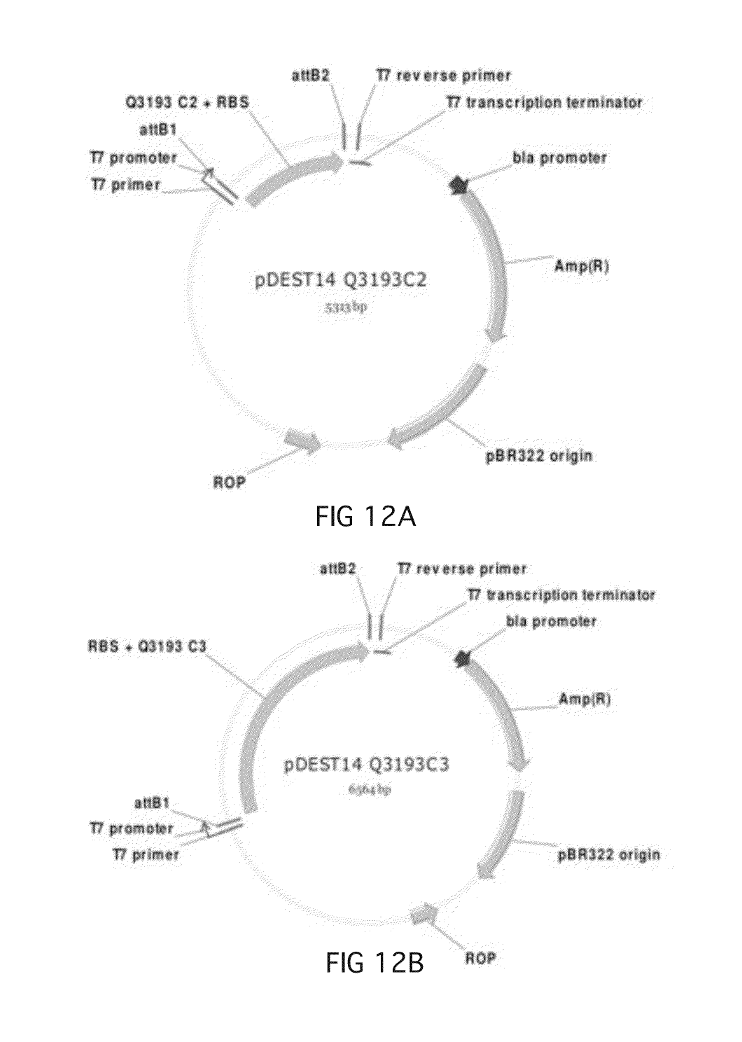

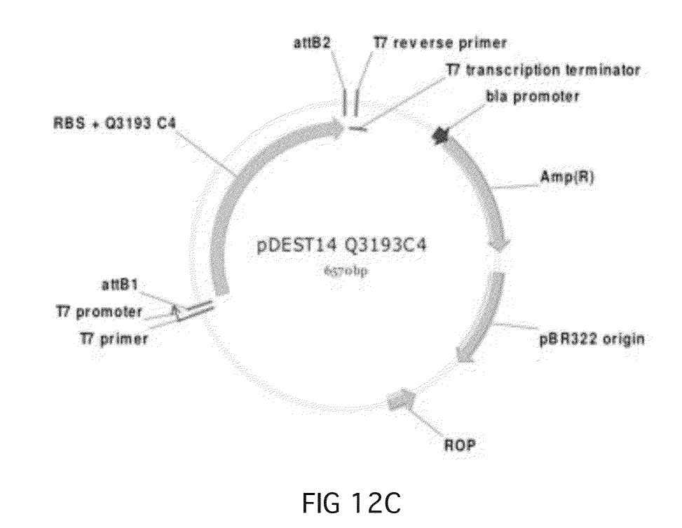

As described below, several gCA constructs based on the Methanosarcina thermophila (www.rcsb.org pdb code 1thj) structural framework were engineered, expressed in E. coli, purified, and used to assemble nanostructures that were characterized using electron microscopic molecular imaging methods. For expression, synthetic gene constructs were incorporated in BL21 STAR (DE3)pLysS expression vectors (FIGS. 12A through 12C). All sequences for synthesized genes were verified after transformation into E. coli.

### Example 1

### EXP14Q3193C2 Expression and Purification of Engineered Trivalent gCA Trimer

Table 1 shows the amino acid sequence (Sequence 1) of an engineered construct based on the 1thj gCA from Methanosarcina thermophila. The construct is a C3 symmetric, 3-subunit, enzyme composed of three identical polypeptide chains. Each subunit of the synthesized protein incorporates two mutations (Asp70 to Cys and Tyr 200 to Cys) to form sites for biotinylation allowing subsequent cross-linking with streptavidin tetramers. In addition, Cys 148 was changed to Ala in each subunit (the amino acid residue numbering follows that assigned to the native polypeptide). In addition, a poly-Histidine sequence was appended to the C-terminus of the polypeptide chain. The assembled trimeric gCA corresponds to the schematic shown in FIG. 7A and consequently forms a structure able to make trivalent interactions with 3 streptavidin tetramers.

The designed sequence was incorporated into a gene sequence and expression vector EXP14Q3193C2 (FIG. 12A) optimized for expression in E. coli. The gene nucleotide sequence for the synthetic sequence EXP14Q3193C2 incorporated into the EXP14Q3193C2 expression vector was:

(SEQ ID NO: 3)
GaaggagatatacatATGCAAGAGATTACCGTTGACGAATTTAGCAATA
TCCGTGAAAACCCGGTTACCCCGTGGAACCCGGAACCGAGCGCCCCCGG
TTATTGACCCGACCGCCTATATTGACCCGGAAGCAAGCGTGATTGGTGA
AGTTACGATTGGCGCAAATGTTATGGTTAGCCCGATGGCGAGCATTCGC
AGCGATGAAGGTATGCCGATTTTTGTGGGTTGTCGTAGCAATGTTCAAG
ATGGTGTTGTCCTGCACGCACTGGAAACGATTAATGAAGAAGGTGAACC
GATTGAAGATAATATTGTTGAAGTTGATGGCAAAGAATACGCAGTTTAT
ATTGGTAATAATGTTAGCCTGGCCCATCAGAGCCAAGTCCACGGTCCGG
CCGCAGGCGATGATACGTTTATTGGCATGCAAGCGTTCGTTTTTAAAAG
CAAAGTGGGTAATAATGCAGTTCTGGAACCGCGTAGCGCAGCGATTGGT
GTCACGATCCCGGATGGTCGCTATATCCCGGCCGGTATGGTCGTTACCA
GCCAAGCAGAAGCAGACAAACTGCCGGAAGTCACCGATGATTACGCCTA
TAGCCATACCAATGAAGCCGTTGTTTGTGTGAATGTTCATCTGGCGGAA
GGTTACAAAGAAACGATTGAAGGCCGTCATCACCACCACCCACCACTAA
gacccagctttcttgtacaaagtggtcccc.

### EXP14Q3193C2 Expression Experiments:

E. coli cells BL21 Star™ (DE3) pLysS with expression vector EXP14Q3193C2 (FIG. 12A) were cultured in 50 mL Terrific Broth supplemented with 0.1 mg/mL ampicillin and 0.034 mg/mL chloramphenicol. The culture was grown overnight at 37° C. to an OD₆₀₀ of 5.53. 0.9 mL was used to inoculate a second culture of 50 mL Terrific Broth supplemented with 0.1 mg/mL ampicillin and 0.034 mg/mL chloramphenicol. The culture was grown overnight at 37° C. to an OD₆₀₀ of 0.807, induced with 0.4 mM IPTG and supplemented with 0.5 mM ZnSO₄, then grown for 4 hours at 25° C. to an OD₆₀₀ of 2.69. 0.6 g of cells were collected by low speed centrifugation.

In a second batch, E. coli cells BL21 Star™ (DE3) pLysS with expression vector EXP14Q3193C2 were cultured in 50 mL Terrific Broth supplemented with 0.1 mg/mL ampicillin and 0.034 mg/mL chloramphenicol. The culture was grown overnight at 37° C. to an OD600 of 5.53. 0.9 mL was used to inoculate a second culture of 50 mL Terrific Broth supplemented with 0.1 mg/mL ampicillin and 0.034 mg/mL chloramphenicol. The culture was grown overnight at 37° C. to an OD600 of 0.807, induced with 0.4 mM IPTG and supplemented with 0.5 mM ZnSO4, then grown for 20 hours at 25° C. to an OD600 of 20.97. 2.0 g of cells were collected by low speed centrifugation.

In a third batch E. coli cells BL21 Star™ (DE3) pLysS with expression vector EXP14Q3193C2 were cultured in 50 mL Luria-Bertani broth supplemented with 0.1 mg/mL ampicillin and 0.034 mg/mL chloramphenicol. The culture was grown overnight at 37° C. to an OD600 of 5.53. 0.9 mL was used to inoculate a second culture of 50 mL Luria-Bertani Broth supplemented with 0.1 mg/mL ampicillin and 0.034 mg/mL chloramphenicol. The culture was grown overnight at 37° C. to an OD600 of 0.753, induced with 0.4 mM IPTG and supplemented with 0.5 mM ZnSO4, then grown for 4 hours at 25° C. to an OD600 of 3.23. 0.8 g of cells were collected by low speed centrifugation.

In a fourth batch E. coli cells BL21 Star™ (DE3) pLysS with expression vector EXP14Q3193C2 were cultured in 50 mL Luria-Bertani broth supplemented with 0.1 mg/mL ampicillin and 0.034 mg/mL chloramphenicol. The culture was grown overnight at 37° C. to an OD600 of 5.53. 0.9 mL was used to inoculate a second culture of 50 mL Luria-Bertani broth supplemented with 0.1 mg/mL ampicillin and 0.034 mg/mL chloramphenicol. The culture was grown overnight at 37° C. to an OD600 of 0.753, induced with 0.4 mM IPTG and supplemented with 0.5 mM ZnSO4, then grown for 20 hours at 25° C. to an OD600 of 23.64. 2.4 g of cells were collected by low speed centrifugation.

Initial expression levels were evaluated using PAGE electrophoresis and Western blots using an anti-His tag antibody to identify the expressed protein product.

### EXP14Q3193C2 Protein Purification:

Following initial expression experiments, fermentations were scaled to the 16 liter scale using standard laboratory scale fermentation equipment under conditions that produced the best expression results in the initial expression experiments. Cells were initially disrupted using sonication, and solids spun down using centrifugation. The resulting supernatant was heated for 20 minutes at 55 deg C., causing precipitation of most of the endogeneously expressed E. coli proteins, but leaving the thermostable engineered construct in solution. Following centrifugation to remove denatured E. coli proteins, the construct protein present in the resulting supernatant was immobilized on a Ni-NTA resin chromatography column, and finally eluted at >95% pure form using 0.25 M imidazole solution. The engineered protein construct was monitored throughout the process using SDS PAGE and/or non-denaturing PAGE followed by western blotting using an anti-His Tag antibody. Additional ion exchange and hydrophobic chromatography showed that the expressed construct behaved nearly identically to the native protein (Alber & Ferry 1996), indicating preservation of native structure and thermal stability of the engineered trimeric construct. Construct recovery levels generally ranged from 5 to 10 mgs per liter of expression fermentation. Correctness of construct expression was confirmed using mass spectroscopy.

### EXP14Q3193C2 Protein Biotinylation:

Covalent attachment of biotin groups to the engineered constructs was performed using cysteine-reactive biotinylation reagents. Best results were obtained with PEG-Linked maleamide reagents (Biotin-d®PEG3-MAL, Quanta Biodesign Limited). Construct biotinylation was monitored both by measuring the loss of reactive cysteines on the construct using Ellman's reagent (Riddles et al. 1983) and measurement of HABA displacement from streptavidin by the biotinylated protein (Green 1965). Alternately, biotinylation reaction progress could be spectroscopically monitored for some reagents by measuring release of a pyridine-2-thione leaving group of the biotinylation reagent. Biotinylation extents of >95% were preferred for gCA constructs used in nanostructure formation.

### EXP14Q3193C2 Hexagonal Nanostructure Formation:

Streptavidin-linked nanostructures were formed on 2D surfaces (FIGS. 13A through 13H). FIG. 13A shows a vessel 1301 containing an aqueous solution, on the surface of which is formed a monolayer consisting of a mixture of lipids 1302 and lesser amount of lipids 1303 that are functionalized on their head group with a Ni-NTA group. For example, dioleoyl phosphatidylcholine can be used as the major monolayer component and Ni-2-(bis-carboxymethyl-amino)-6-[2-(1,3)-di-O-oleyl-glyceroxy)-acetyl-amino]hexanoic acid (Ni-NTA-DOGA) can be used as the Ni-containing phospholipid. FIG. 13B illustrates the exemplary introduction of a trivalent biotinylated construct shown in plan 1304 and side view 1305. The trivalent node incorporates 3 pair of biotinylation sites 1306, and a terminal poly-Histidine sequence 1307. A solution containing the biotinylated construct is introduced below the surface of the monolayer using a syringe 1308. The biotinylated constructs 1309 attach to the Ni-NTA lipids through interactions formed between the Ni-NTA and the poly-Histidine terminus of the construct. The monolayer is fluid, so that the nodes 1309 are free to diffuse in the plane of the monolayer. FIG. 13C shows the introduction of streptavidin 1310 under the surface of the monolayer using syringe 1311. Typically, the added streptavidin may be saturated with a dye HABA (Green 1965) that binds to the biotin binding sites of streptavidin. Attachments formed between the freely diffusing nodes and streptavidin produce the assembled nanostructure 1312. The displacement of the HABA dye from streptavidin by biotin when the nanostructure is formed causes a color change that can be followed to monitor nanostructure assembly. FIG. 13D shows the assembled nanostructure and monolayer 1313 contacted by a surface 1312 with and affinity for the hydrophobic surface of the monolayer. FIG. 13E shows the assembled nanostructure and monolayer lifted from the liquid and attached to the surface 1314, for example an electron microscope grid. FIG. 13F shows a schematic of a hexagonal nanolattice formed using streptavidin and trivalent nodes. Many different nanostructures can be prepared using this general method, depending on the node valency, use of preassembled components, and order of component addition and assembly.

### EXP14Q3193C2 Hexagonal Nanostructure Electron Microscopy:

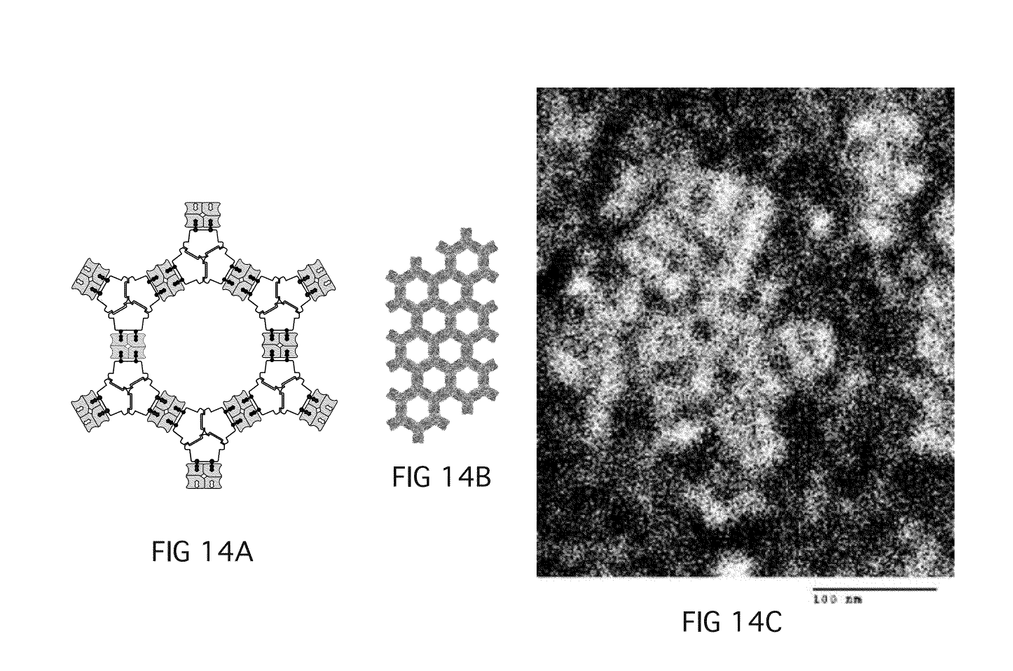

FIG. 14A shows a schematic illustration of a hexagonal lattice formed through the assembly of trivalent biotinylated nodes and streptavidin. FIG. 14B shows a molecular model of the structure based on a trivalent node construct of the Methanosarcina thermophila 1thj gCA structure to the scale of the electron microscope image shown in FIG. 14C. FIG. 14C shows a uranyl acetate negatively stained region of an electron microscope grid showing the formation of regions of hexagonal nanostructure prepared using streptavidin and a trivalent construct of EXP14Q3193C2 (Table 1, Sequence 1), substantially as described in FIGS. 13A through 13H. Images were taken at 50,000× at 100 kV using a Carl Zeiss LEO Omega 912 energy filtered transmission electron microscope (EF-TEM) equipped with a 7.5 mega-pixel Hamamatsu Orca EMCCD camera. The results indicate the ability of the engineered constructs to form 2D hexagonal lattices on monolayer surfaces.

### Example 2

### EXP14Q3193C3 Expression and Purification of Engineered Trivalent Single-Chain gCA

Table 1 shows the amino acid sequence (Sequence 2) of an engineered, trivalent, single chain gCA construct based on the 1thj gCA from Methanosarcina thermophila. The structure incorporates 3 subunits covalently linked with two GGSGGG (Gly-Gly-Ser-Gly-Gly-Gly) (SEQ ID NO: 4) sequences, and with each subunit incorporating a pair of cysteine residues in positions corresponding to the position in the EXP14Q3193C3. The assembled trimeric gCA corresponds to the schematic shown in FIG. 7B and consequently forms a structure able to make trivalent interactions with 3 streptavidin tetramers.

The designed sequence was incorporated into a gene sequence and expression vector EXP14Q3193C3 (FIG. 12B) optimized for expression in E. coli. The gene nucleotide sequence for the synthetic sequence EXP14Q3193C3 incorporated into the EXP14Q3193C3 expression vector was:

(SEQ ID NO: 5)
ggggacaagtttgtacaaaaaagcaggcaccgaaggagatatacatATG
GATGAATTTAGCAATATCCGCGAAAATCCGGTGACCCCGTGGAATCCGG
AACCGAGCGCCCCCGGTTATTGATCCGACGGCATACATCGACCCGGAAG
CCAGCGTGATTGGTGAAGTTACCATCGGCGCCAATGTTATGGTCAGCCC
GATGGCGAGCATCCGCAGCGATGAAGGCATGCCGATCTTTGTGGGCTGT
CGTAGCAATGTGCAGGATGGCGTTGTTCTGCACGCGCTGGAAACCATTA
ATGAAGAAGGCGAACCGATTGAAGACAATATTGTTGAAGTGGACGGTAA
GGAATATGCAGTGTACATCGGTAACAACGTCAGCCTGGCCCATCAGAGC
CAAGTCCATGGTCCGGCCGCCGTGGGCGATGATACCATTGGCATGCAAG
CGTTCGTGTTTAAAAGCAAAGTTGGCAATAATGCAGTTCTGGAACCGCG
CAGCGCGGCGATCGGCGTGACCATTCCGGATGGTCGTTACATCCCGGCC
GGCATGGTGGTCACCAGCCAAGCGGAGGCCGATAAACTGCCGGAAGTCA
CCGATGACTATGCCTATAGCCACACCAATGAGGCCGTCGTGTGCGTGAA
CGTTCATCTGGCCGAAGGTTATAAAGAAACGGGTGGTAGCGGCGGCGGC
GATGAATTTAGCAATATCCGCGAAAATCCGGTGACCCCGTGGAATCCGG
AGCCGAGCGCACCGGTTARRGATCCGACCGCATATATTGATCCGGAGGC
CAGCGTTATCGGCGAAGTTACGATCGCGAATGTTATGGTGAGCCCGATG
GCGAGCATTCGCAGCGATGAGGGTATGCCGATTTTTGTGGGCTGCCGTA
GCAATGTGCAAGATGGTGTGGTCCTGCACGCACTGGAGACGATTAACGA
GGAAGGTGAACCGATCGAGGACAACATTGTCGAAGTGGACGGTAAGGAG
TATGCGGTGTATATCGGCAACAACGTTAGCCTGGCCCACCAGAGCCAGG
TGCACGGCCCGGCAGCAGTGGGCGATGACACGTTTATTGGCATGCAGGC
GTTCGTTTTCAAAAGCAAAGTTGGCAATAACGCAGTTCTGGAACCGCGT
AGCGCAGCGATTGGCGTTACCATCCCGGATGGCCGTTATATCCCGGCCG
GTATGGTCGTTACGCAGGCGGAAGCAGATAAACTGCCGGAAGTTACCGA
TGACTATGCCTATAGCCATACCAATGAGGCAGTTGTTTGTGTCAATGTC
CATCTGGCGGAAGGCTACAAAGAAACGGGTGGTAGCGGTGGCGGTGATG
AATTCAGCAACATCCGTGAAAACCCGGTGACCCCGTGGAACCCGGAACC
GAGCGCGCCGGTCATTGATCCGACCGCATATATCGATCCGGAGGCAAGC
GTCATTGGCGAAGTTACGATTGGCGCCAACGTGATGGTCAGCCCGATGG
CCAGCATCCGCAGCGATGAAGGCATGCCGATTTTTGTTGGTTGCCGTAG
CAACGTTCAGGATGGCGTGGTCCTGCACGCACTGGAAACCATTAACGAA
GAAGAGCCGATTGAAGATAACATCGTTGAGGTCGACGGTAAAGAATATG
CCGTGTATATCGGCAACAACGTTAGCCTGGCCCATCAAAGCCAAGTTCA
TGGTCCGGCCGCGGTTGGTGATGACACGTTCATTGGCATGCAGGCGTTT
GTGTTTAAGAGCAAAGTGGGTAATAATGCCGTTCTGGAGCCGCGCAGCG
CCGCAATCGGCGTCACCATCCCGGACGGTCGCTACATTCCGGCAGGCAT
GGTCGTGACCAGCCAAGCCGAAGCGGACAAACTGCCGGAAGTCACCGAT
GATTAGCATACAGCCACACCAACGAGGCGGTCGTGTGTGTTAATGTGCA
TCTGGCGGAAGGTTATAAAGAAACGATTGAAGGCCGTCATCACCACCAT
CATTGAacccagctttcttgtacaaagtggtgatgatccggctgctaac
aaagcccgaaaggaagctga.

### EXP14Q3193C3 Expression Experiments:

E. coli cells BL21 Star™ (DE3) with expression vector EXP14Q3193C3 (FIG. 12B) were cultured in 50 mL Luria-Bertani broth supplemented with 0.1 mg/mL ampicillin and 0.034 mg/mL chloramphenicol. The culture was grown overnight at 37° C. to an OD₆₀₀ of 6.83. 0.73 mL was used to inoculate a second culture of 50 mL Luria-Bertani broth supplemented with 0.1 mg/mL ampicillin and 0.034 mg/mL chloramphenicol. The culture was grown overnight at 37° C. to an OD₆₀₀ of 0.949, induced with 0.4 mM IPTG and supplemented with 0.5 mM ZnSO₄, then grown for 4 hours at 25° C. to an OD₆₀₀ of 2.78. 0.6 g of cells were collected by low speed centrifugation.

In a second batch, E. coli cells BL21 Star™ (DE3) with expression vector EXP14Q3193C3 were cultured in 50 mL Luria-Bertani broth supplemented with 0.1 mg/mL ampicillin and 0.034 mg/mL chloramphenicol. The culture was grown overnight at 37° C. to an OD₆₀₀ of 6.83. 0.73 mL was used to inoculate a second culture of 50 mL Luria-Bertani broth supplemented with 0.1 mg/mL ampicillin and 0.034 mg/mL chloramphenicol. The culture was grown overnight at 37° C. to an OD₆₀₀ of 0.949, induced with 0.4 mM IPTG and supplemented with 0.5 mM ZnSO₄, then grown for 20 hours at 25° C. to an OD₆₀₀ of 4.49. 0.8 g of cells were collected by low speed centrifugation.

In a third batch E. coli cells BL21 Star™ (DE3) with expression vector EXP14Q3193C3 were cultured in 50 mL Terrific broth supplemented with 0.1 mg/mL ampicillin and 0.034 mg/mL chloramphenicol. The culture was grown overnight at 37° C. to an OD₆₀₀ of 6.83. 0.73 mL was used to inoculate a second culture of 50 mL Terrific broth supplemented with 0.1 mg/mL ampicillin and 0.034 mg/mL chloramphenicol. The culture was grown overnight at 37° C. to an OD₆₀₀ of 0.796, induced with 0.4 mM IPTG and supplemented with 0.5 mM ZnSO₄, then grown for 4 hours at 25° C. to an OD₆₀₀ of 3.94. 0.7 g of cells were collected by low speed centrifugation.

In a fourth batch E. coli cells BL21 Star™ (DE3) with expression vector EXP14Q3193C3 were cultured in 50 mL Terrific broth supplemented with 0.1 mg/mL ampicillin and 0.034 mg/mL chloramphenicol. The culture was grown overnight at 37° C. to an OD600 of 6.83. 0.73 mL was used to inoculate a second culture of 50 mL Terrific broth supplemented with 0.1 mg/mL ampicillin and 0.034 mg/mL chloramphenicol. The culture was grown overnight at 37° C. to an OD600 of 0.89, induced with 0.4 mM IPTG and supplemented with 0.5 mM ZnSO₄, then grown for 20 hours at 25° C. to an OD600 of 17.52. 1.9 g of cells were collected by low speed centrifugation.

In a fifth batch E. coli cells BL21 Star™ (DE3) pLysS with expression vector EXP14Q3193C3 were cultured in 50 mL Luria-Bertani broth supplemented with 0.1 mg/mL ampicillin and 0.034 mg/mL chloramphenicol. The culture was grown overnight at 37° C. to an OD₆₀₀ of 5.63. 0.89 mL was used to inoculate a second culture of 50 mL Luria-Bertani broth supplemented with 0.1 mg/mL ampicillin and 0.034 mg/mL chloramphenicol. The culture was grown overnight at 37° C. to an OD₆₀₀ of 0.905, induced with 0.4 mM IPTG and supplemented with 0.5 mM ZnSO₄, then grown for 4 hours at 25° C. to an OD₆₀₀ of 2.92. 0.6 g of cells were collected by low speed centrifugation.

In a sixth batch E. coli cells BL21 Star™ (DE3) pLysS with expression vector EXP14Q3193C3 were cultured in 50 mL Luria-Bertani broth supplemented with 0.1 mg/mL ampicillin and 0.034 mg/mL chloramphenicol. The culture was grown overnight at 37° C. to an OD₆₀₀ of 5.63. 0.89 mL was used to inoculate a second culture of 50 mL Luria-Bertani broth supplemented with 0.1 mg/mL ampicillin and 0.034 mg/mL chloramphenicol. The culture was grown overnight at 37° C. to an OD₆₀₀ of 0.905, induced with 0.4 mM IPTG and supplemented with 0.5 mM ZnSO₄, then grown for 20 hours at 25° C. to an OD₆₀₀ of 3.62. 0.8 g of cells were collected by low speed centrifugation.

In a seventh batch E. coli cells BL21 Star™ (DE3) pLysS with expression vector EXP14Q3193C3 were cultured in 50 mL Terrific broth supplemented with 0.1 mg/mL ampicillin and 0.034 mg/mL chloramphenicol. The culture was grown overnight at 37° C. to an OD₆₀₀ of 5.63. 0.89 mL was used to inoculate a second culture of 50 mL Terrific broth supplemented with 0.1 mg/mL ampicillin and 0.034 mg/mL chloramphenicol. The culture was grown overnight at 37° C. to an OD₆₀₀ of 0.796, induced with 0.4 mM IPTG and supplemented with 0.5 mM ZnSO₄, then grown for 4 hours at 25° C. to an OD₆₀₀ of 3.87. 1.3 g of cells were collected by low speed centrifugation.

In an eighth batch E. coli cells BL21 Star™ (DE3) pLysS with expression vector EXP14Q3193C3 were cultured in 50 mL Terrific broth supplemented with 0.1 mg/mL ampicillin and 0.034 mg/mL chloramphenicol. The culture was grown overnight at 37° C. to an OD₆₀₀ of 5.63. 0.89 mL was used to inoculate a second culture of 50 mL Terrific broth supplemented with 0.1 mg/mL ampicillin and 0.034 mg/mL chloramphenicol. The culture was grown overnight at 37° C. to an OD₆₀₀ of 0.796, induced with 0.4 mM IPTG and supplemented with 0.5 mM ZnSO₄, then grown for 20 hours at 25° C. to an OD₆₀₀ of 18.22. 1.9 g of cells were collected by low speed centrifugation.

In a production run, E. coli cells BL21 Star™ (DE3) pLysS with expression vector EXP14Q3193C3 were cultured in 375 mL Terrific broth supplemented with 0.1 mg/mL ampicillin and 0.034 mg/mL chloramphenicol. The culture was grown overnight at 37° C. to an OD₆₀₀ of 4.276. The culture was used to inoculate a second culture of 16 L Terrific broth supplemented with 0.1 mg/mL ampicillin and 0.034 mg/mL chloramphenicol. The culture was grown overnight at 37° C. with 30% dissolved oxygen and 400-550 rpm to an OD₆₀₀ of 1.053, induced with 0.4 mM IPTG and supplemented with 0.5 mM ZnSO₄, then grown for 19.75 hours at 25° C. to an OD₆₀₀ of 7.34. 182.5 g of cells were collected by low speed centrifugation.

### EXP14Q3193C3 Protein Purification:

The single-chain trivalent, engineered gCA was isolated from the collected E. coli cells generated from a 16 L production run using expression vector EXP14Q3193C3 as follows. 10 grams of E. coli cells with EXP14Q3193C3 were suspended in 20 mL 50 mM KPO₄ buffer pH 6.8, 30 mg lysozyme, 1 mg DNase I, and one pellet EDTA-free protease inhibitors (Roche). The suspension was held at 4° C. and stirred for 1 hour, then sonicated in 3 sets of 30 1-second pulses. The suspension was centrifuged at 12500×g for 20 min. The soluble portion was subjected to column chromatography on Q-Sepharose equilibrated with 50 mM KPO₄ buffer pH 6.8, 0.001 mM ZnSO₄. Node protein was eluted by a linear gradient between 50 mM KPO₄ buffer pH 6.8, 0.001 mM ZnSO₄ and 50 mM KPO₄ buffer pH 6.8, 0.001 mM ZnSO₄, 1 M NaCl. Node protein fractions were identified by PAGE SDS analyses, then pooled and loaded onto a Phenyl-Sepharose chromatography column equilibrated with 50 mM KPO₄ buffer pH 6.8, 0.001 mM ZnSO₄, 1 M NaCl. Node protein was eluted from the column by a linear gradient between 50 mM KPO₄ buffer pH 6.8, 0.001 mM ZnSO₄, 1 M NaCl and 50 mM KPO₄ buffer pH 6.8, 0.001 mM ZnSO₄. Node protein fractions identified by PAGE SDS analyses were combined and dialyzed against 2 changes of 25 mM NaPO₄ buffer pH 8.0 with each change corresponding to at least 10× node protein volume. Dialyzed node protein was mixed with 3 mL Ni agarose resin equilibrated with 25 mM NaPO₄ buffer pH 8.0, then reacted for 18 hours with rocking at 4° C. The resin was washed with twice with 15 mL 25 mM NaPO₄ buffer pH 8.0, then the node protein was eluted with 25 mM NaPO₄ buffer pH 8.0, 250 mM imidazole.

A second, alternative isolation procedure was carried out in a similar manner, except that the Ni agarose resin was used before the Q-sepharose and phenyl-Sepharose chromatographic steps. A third, alternative isolation procedure was carried out in a similar manner, except that the E. coli cells were disrupted by addition of nonionic detergent (B-PER ThermoScientific) instead of by addition of lysozyme followed by stirring and sonication.

Following isolation, construct expression was confirmed using MALDI mass spectroscopy.

### EXP14Q3193C3 Trivalent Single-Chain gCA Construct Microscopy:

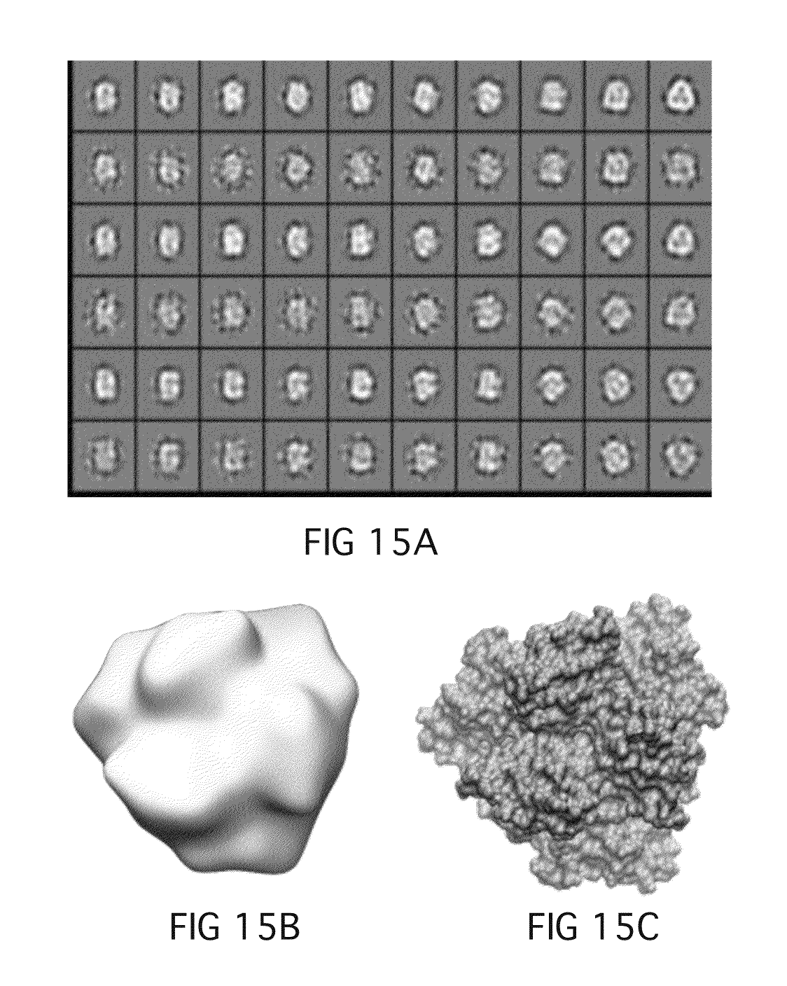

FIG. 15A shows 60 uranyl acetate, negatively stained, electron microscope images of isolated molecules of the trivalent single-chain node construct of the Methanosarcina thermophile 1thj gCA (Table 1, Sequence 2). FIG. 15B shows a computer-averaged reconstruction of the images based on mathematical correlation and superposition. FIG. 15 C shows the molecular surface computed from Methanosarcina thermophile 1thj gCA engineered structure atomic coordinates. The correspondence of FIGS. 15B and 15C clearly demonstrates the preservation of structural organization in the gCA engineered single-chain construct. Images were taken at 100,000× at 200 kV using a JEOL 2100F electron microscope equipped with a Tietz 2kX2K CCD camera. Images were processed for 3D reconstruction using the SerialEM computational program system for electron microscopy imaging.

### Example 3

### EXP14Q3193C4 Expression and Purification of Engineered Bivalent Single-Chain gCA

Table 1 shows the amino acid sequence (Sequence 3) of an engineered, bivalent, single chain gCA construct based on the 1thj gCA from Methanosarcina thermophila. The structure incorporates 3 subunits covalently linked with two GGSGGG (Gly-Gly-Ser-Gly-Gly-Gly) (SEQ ID NO: 4) sequences, but with only two subunits incorporating pairs of cysteine residues in positions corresponding to the positions in EXP14Q3193C3. The assembled trimeric gCA corresponds to the schematic shown in FIG. 7C and consequently forms a structure able to make bivalent interactions with 2 streptavidin tetramers.

The designed sequence was incorporated into a gene sequence and expression vector EXP14Q3193C4 (FIG. 12C) optimized for expression in E. coli. The gene nucleotide sequence for the synthetic sequence EXP14Q3193C3 incorporated into the EXP14Q3193C3 expression vector was:

(SEQ ID NO: 6)
cgatgcgtccggcgtagaggatcgagatctcgatcccgcgaaattaata
cgactcactatagggagaccacaacggtttccctctagatcacaagttt
gtacaaaaaagcaggcaccgaaggagatatacatATGGATGAATTTAGC
AATATTCGCGAAAACCCGGTTACCCCGTGGAACCCGGAACCGAGCGCGC
CGGTTATCGACCCGACGGCCTACATTGATCCGGAGGCAAGCGTGATTGG
TGAAGTGACGATTGGTGCAAATGTCATGGTGAGCCCGATGGCGAGCATT
CGTAGCGATGAAGGTATGCCGATTTTCGTTGGTTGTCGTAGCAATGTTC
AAGATGGTGTTGTTCTGCACGCCCTGGAAACCATTAATGAAGAAGGTGA
GCCGATTGAAGACAACATCGTTGAAGTTGATGGTAAAGAATACGCGGTT
TATATCGGCAACAACGTCAGCCTGGCACATCAGAGCCAAGTTCATGGTC
CGGCAGCAGTGGGCGATGATACGATTGGTATGCAAGCATTCGTTTTTAA
AAGCAAAGTTGGTAATAATGCAGTTCTGGAACCGCGCAGCGCAGCAATT
GGTGTTACCATTCCGGATGGTCGTTATATCCCGGCCGGTATGGTGGTGA
CGAGCCAGGCGGAAGCAGATAAACTGCCGGAAGTGACGGATGATTATGC
CTATAGCCATACCAATGAAGCAGTCGTGTGTGTTAACGTGCACCTGGCC
GAAGGTTACAAAGAAACGGGCGGTGGTAGCGGTGGCGGCGATGAATTTA
GCAATACCGTGAAAACCCGGTTACCCGTGGAATCCGGAACCGAGCGCAC
CGGTTATTGATCCGACGGCATATATCGACCCGGAGGCAAGCGTGATTGG
CGAAGTTACGGGCGCAAATGTGATGGTTAGCCCGATGGCCAGCATTCGT
AGCGATGAAGGCATGCCGATTTTTGTGGCTGCCGCAGCAATGTTCAAGA
TGGTGTTGTCCTGCACGCACTGGAGACCATCAATGAAGAAGGTGAACCG
ATTGAAGATAACATCGTCGAAGTTGACGGCAAAGAATATGCGGTGTATA
TTGGCAATAATGTCAGCCTGGCACATCAAAGCCAAGTTCACGGTCCGGC
AGCAGTGGGCGATGATACCTTTATTGGCATGCAAGCGTTTGTTTTCAAA
AGCAAAGTCGGCAATAATGCAGTTCTGGAACCGCGCGCAGCGCAGCGAT
TGGCGTCACGATCCCGGATGGTCGTTATATTCCGGCCGGCATGGTGGTG
AGCCAGGCAGAAGCAGATAAACTGCCGGAAGTGACCGATGACTATGCCT
ATAGCCATACGAACGAAGCCGTTGTTTGCGTGAACGTGCACCTGGCAGA
AGGCTACAAAGAAACCGGTGGTGGCAGCGGCGGCGGTGATGAATTCAGC
AATATTCGCGAAAATCCGGTCACCCCGTGGAATCCGGAACCGAGCGCCC
CGGTCATTGACCCGACGGCATATATTGATCCGGAAGCAAGCGTTATTGG
TGAAGTTACGATTGGTGCAAACGTGATGGTGAGCCCGATGGCGAGCATT
CGCAGCGATGAGGGCATGCCGATTTTTGTGGGCGATCGCAGCAATGTTC
AAGATGGTGTTGTCCTGCACGCCCTGGAAACCATCAATGAAGGCGAACC
GATTGAAGACAATATTGTGGAAGTCGATGGTAAAGAATACGCAGTCTAT
ATTGGTAATAATGTTAGCCTGGCACATCAGAGCCAAGTCCACGGTCCGG
CCGCAGTGGGTGATGACAGTTTATTGGTATGCAAGCATTTGTGTTTAAA
AGCAAAGTCGGTAACAATGCAGTTCTGGAACCGCGCAGCGCAGCAATCG
GCGTTACGATCCCGGATGGCCGTTATATCCCGGCGGGTATGGTGGTTAC
GAGCCAAGCAGAAGCGGATAAACTGCCGGAAGTTACGGATGATTATGCC
TATAGCCATACGAACGAAGCGGTTGTCTACGTTAACGTGCATCTGGCGG
AGGGTTACAAAGAAACGATTGAGGGTCATCATCACCATCATCATTGAaa
cccagctttc.

### EXP14Q3193C3 Expression Experiments:

E. coli cells BL21 Star™ (DE3) with expression vector EXP14Q3193C4 (FIG. 12C) were cultured in 50 mL Terrific broth supplemented with 0.1 mg/mL ampicillin and 0.034 mg/mL chloramphenicol. The culture was grown overnight at 37° C. to an OD₆₀₀ of 6.04. 0.83 mL was used to inoculate a second culture of 50 mL Terrific broth supplemented with 0.1 mg/mL ampicillin and 0.034 mg/mL chloramphenicol. The culture was grown overnight at 37° C. to an OD₆₀₀ of 0.963, induced with 0.4 mM IPTG and supplemented with 0.5 mM ZnSO₄, then grown for 4 hours at 25° C. to an OD₆₀₀ of 7.57. 0.7 g of cells were collected by low speed centrifugation.

In a second batch E. coli cells BL21 Star™ (DE3) with expression vector EXP14Q3193C4 were cultured in 50 mL Terrific broth supplemented with 0.1 mg/mL ampicillin and 0.034 mg/mL chloramphenicol. The culture was grown overnight at 37° C. to an OD₆₀₀ of 6.04. 0.83 mL was used to inoculate a second culture of 50 mL Terrific broth supplemented with 0.1 mg/mL ampicillin and 0.034 mg/mL chloramphenicol. The culture was grown overnight at 37° C. to an OD₆₀₀ of 0.963, induced with 0.4 mM IPTG and supplemented with 0.5 mM ZnSO₄, then grown for 20 hours at 25° C. to an OD₆₀₀ of 22.8. 2.1 g of cells were collected by low speed centrifugation.

### EXP14Q3193C4 Protein Purification:

Approximately 2 g of E. coli cells expressing the EXP14Q3193C4 vector were disrupted using sonication, and solids spun down using centrifugation. The resulting supernatant was heated for 20 minutes at 55 deg C., causing precipitation of most of the endogeneously expressed E. coli proteins, but leaving the thermostable engineered construct in solution. Following centrifugation to remove denatured E. coli proteins, the construct protein present in the resulting supernatant was immobilized on a Ni-NTA resin chromatography column, and finally eluted at >95% pure form using 0.25 M imidazole solution. The engineered protein construct was monitored throughout the process using SDS PAGE and/or non-denaturing PAGE followed by western blotting using and anti-His Tag antibody. Additional ion exchange and hydrophobic chromatography showed that the expressed construct behaved nearly identically to the native protein (Alber & Ferry 1996), indicating preservation of native structure and thermal stability of the engineered trimeric construct. Construct recovery levels generally ranged from 5 to 10 mgs per liter of expression fermentation broth and correctness of construct expression confirmed using protein mass spectroscopy.

### EXP14Q3193C4 Protein Biotinylation:

Covalent attachment of biotin groups to the engineered constructs was performed using cysteine-reactive biotinylation reagents. Best results were obtained with PEG-Linked maleamide reagents (Biotin-d®PEG3-MAL, Quanta Biodesign Limited). Construct biotinylation was monitored both by measuring the loss of reactive cysteines on the construct using Ellman's reagent (Riddles et al. 1983) and measurement of HABA displacement from streptavidin by the biotinylated protein (Green 1965). Alternately, biotinylation reaction progress could be spectroscopically monitored for some reagents by measuring release of a pyridine-2-thione leaving group of the biotinylation reagent. Biotinylation extents of >95% were preferred for gCA constructs used in nanostructure formation.

### EXP14Q3193C4 Hexagon Nanostructure Formation:

Streptavidin-linked nanostructures were formed on 2D surfaces using an apparatus as shown in FIGS. 13A through 13H. The only departure from the method of FIGS. 13A through 13H involved the addition of the single-chain bivalent gCA construct FIG. 13G during the assembly process, instead of the trivalent trimer construct 1304 in FIG. 13B. Final assembly produces a nanohexagon construct FIG. 13H constructed of a combination of streptavidin and single-chain bivalent nodes. Many different nanostructures can be prepared using this general method, depending on the node valency, use of preassembled components, and order of component addition and assembly.

### EXP14Q3193C4 Hexagon Nanostructure Electron Microscopy:

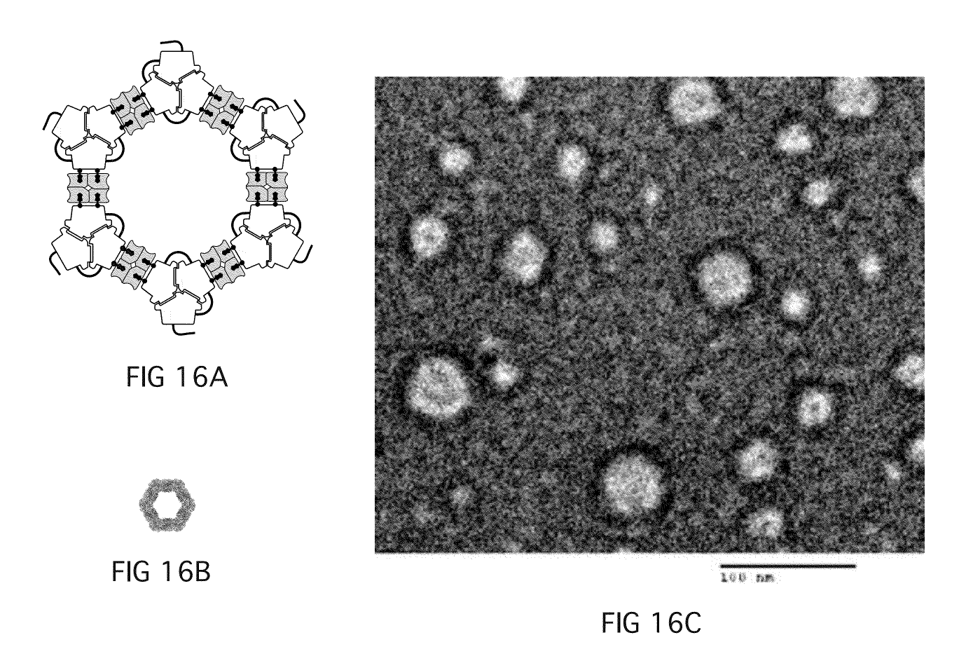

FIG. 16A shows a schematic illustration of a hexagon nanostructure formed through the assembly of bivalent single-chain biotinylated nodes and streptavidin. FIG. 16B shows a molecular model of the nanohexagon structure based on a bivalent single-chain node construct of the Methanosarcina thermophila 1thj gCA structure to the scale of the electron microscope image shown in FIG. 16C. FIG. 16C shows a negatively stained region of an electron microscope grid with nanohexagons prepared using streptavidin and a bivalent single-chain construct of the Methanosarcina thermophila 1thj gCA, substantially as described in FIGS. 13A through 13H. Images were taken at 50,000× at 100 kV using a Carl Zeiss LEO Omega 912 energy filtered transmission electron microscope (EF-TEM) equipped with a 7.5 mega-pixel Hamamatsu Orca EMCCD camera. The results indicate the ability of the engineered constructs to form 2D hexagons on monolayer surfaces.

Thus, all of the proteins expressed by the vectors EXP14Q3193C2 (Example 1), EXP14Q3193C3 (Example 2), and EXP14Q3193C4 (Example 2), could be and were expressed in E. coli. Subsequent protein isolation experiments showed that the expressed constructs behaved with native-like properties and retained a compact folded and soluble state, all consistent with the preservation of gCA enzyme structure and function. Electron microscope examination of both assembled nanostructures (Examples 1 and 3) as well as imaging of isolated single-chain gCA constructs (Example 2) confirmed expectations regarding geometry and dimensions of engineered constructs and nanostructures assembled on 2D surfaces.

### Example 4

### Engineered Ultrastable Trimeric gCA

Table 1 (Sequence 4) shows the amino acid sequence of an engineered, trimeric gCA construct based on the 1v3w gCA from Pyrococcus horikoshii OT3. Each polypeptide chain has been extended on its C-terminus with a poly-Histidine sequence to facilitate isolation and allow immobilization on a Ni-NTA functionalized surface. The sequence shown corresponds to the schematic shown in FIG. 3A.

### Example 5

### Engineered Ultrastable Trimeric Trivalent gCA

Table 1 (Sequence 5) shows the amino acid sequence of an engineered, trimeric gCA construct based on the 1v3w gCA from Pyrococcus horikoshii OT3. Each polypeptide chain sequence has been modified through conversion to cysteine residues at positions indicated by bold C in Table 1-Sequence 5 to allow biotinylation at locations on the gCA trimer surface that are pair-wise complementary to binding sites on streptavidin. In addition, each polypeptide chain has been extended on its C-terminus with a poly-Histidine sequence to facilitate isolation and allow immobilization on a Ni-NTA functionalized surface. The sequence shown corresponds to the schematic shown in FIG. 7A.

### Example 6

### Engineered Ultrastable Single-Chain gCA

Table 1-Sequence 6 shows the amino acid sequence of an engineered, single-chain gCA construct based on the 1v3w gCA from Pyrococcus horikoshii OT3. The structure incorporates 3 subunits covalently linked with two GSGGS (Gly-Ser-Gly-Gly-Ser) (SEQ ID NO: 7) sequences, forming a single continuous polypeptide chain. In addition, the linked polypeptide chain has been extended on its C-terminus with a poly-Histidine sequence to facilitate isolation and allow immobilization on a Ni-NTA functionalized surface. The sequence shown corresponds to the schematic shown in FIG. 3B.

### Example 7

### Engineered Ultrastable Monovalent Single-Chain gCA

Table 1-Sequence 7 shows the amino acid sequence of an engineered, trimeric gCA construct based on the 1v3w gCA from Pyrococcus horikoshii OT3. The structure incorporates 3 subunits covalently linked with two GSGGS (Gly-Ser-Gly-Gly-Ser) (SEQ ID NO: 7) sequences, forming a single continuous polypeptide chain. One polypeptide chain sequence has been modified through conversion to cysteine residues at positions indicated by bold C in Table 1-Sequence 7 to allow biotinylation at locations on one gCA trimer surface that are pair-wise complementary to binding sites on streptavidin. In addition, the linked polypeptide chain has been extended on its C-terminus with a poly-Histidine sequence to facilitate isolation and allow immobilization on a Ni-NTA functionalized surface. The sequence shown is a variation of the schematic shown in FIG. 7D.

### Example 8

### Engineered Ultrastable Single-Chain gCA Incorporating Biotinylation Sequence

Table 1-Sequence 8 shows the amino acid sequence of an engineered, trimeric gCA construct based on the 1v3w gCA from Pyrococcus horikoshii OT3. The structure incorporates 3 subunits covalently linked with two GSGGS (Gly-Ser-Gly-Gly-Ser) (SEQ ID NO: 7) sequences, forming a single continuous polypeptide chain. In addition, the linked polypeptide chain has been extended on its C-terminus with a sequence allowing enzymatic biotinylation in suitable E. coli or other heterologous (e.g. yeast) expression systems.

This application hereby incorporates by reference the following in their entirety: U.S. Provisional Application Ser. No. 60/996,089 (filed Oct. 26, 2007); International Application Serial Number PCT/US2008/012174 (filed Oct. 27, 2008, published as WO/2009/055068 on Apr. 30, 2009); U.S. Provisional Application Ser. No. 61/173,114 (filed Apr. 27, 2009); U.S. application Ser. No. 12/766,658 (filed Apr. 23, 2010, published as US2010-0329930 on Dec. 30, 2010); U.S. Provisional Application Ser. No. 61/136,097 (filed Aug. 12, 2008); U.S. application Ser. No. 12/589,529 (filed Apr. 27, 2009, published as US2010-0256342 on Oct. 7, 2010); international application Serial Number PCT/US2009/053628 (filed Aug. 13, 2009, published as WO/2010/019725 on Feb. 18, 2010); U.S. Provisional Application Ser. No. 61/246,699 (filed Sep. 29, 2009); U.S. application Ser. No. 12/892,911 (filed Sep. 28, 2010, published as US2011-0085939 on Apr. 14, 2011); U.S. Provisional Application Ser. No. 61/177,256 (filed May 11, 2009); International Application Serial Number PCT/US2010/034248 (filed May 10, 2010, published as WO/2010/132363 on Nov. 18, 2010); U.S. application Ser. No. 13/319,989 (filed Nov. 10, 2011); U.S. Provisional Application Ser. No. 61/444,317 (filed Feb. 18, 2011); U.S. application Ser. No. 13/398,820 (filed Feb. 16, 2012); and U.S. Provisional Application No. 61/611,205 (filed Mar. 15, 2012). All documents cited herein or cited in any one of the patent applications, published patent applications, and patents incorporated by reference are hereby incorporated by reference in their entirety.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

Alber B E, Colangelo C M, Dong J. Stålhandske C M V, Baird T T, |Tu C. Fierke C A, Silverman D N, Scott R A, Ferry J G. Kinetic and Spectroscopic Characterization of the Gamma-Carbonic Anhydrase from the Methanoarchaeon Methanosarcina thermophile, (1999) Biochemistry 38, 13119-13128
Barat B, Wu A M, Metabolic biotinylation of recombinant antibody by biotin ligase retained in the endoplasmic reticulum Biomol Eng (2007) 24:283-291.
Borchert, M, Saunders P. “Heat-stable carbonic anhydrases and their use” (2010) U.S. Pat. No. 7,803,575
Case D A, Cheatham T E, Darden T, Gohlke H, Luo R, Merz K M, Onufriev A, Simmerling C, Wang B, Woods R. “The Amber biomolecular simulation programs” J Comput Chem (2005) 26:1668-1688.
Chapman-Smith A, Cronan J E J. Molecular Biology of biotin attachment to proteins J Nutr (1999) 129:477 S-484S.
Finzel B C, Kimatian S, Ohlendorf D H, Wendoloski J J, Levitt M, Salemme F R. Molecular Modeling with Substructure Libraries Derived from Known Protein Structures In Crystallographic and Modeling Methods in Molecular Design (S Ealick & C Bugg eds.) Springer Verlag, New York (1990) pp. 175-189.
Ge J J, Cowan R M, Tu C K, McGregor M L, Trachtenberg M C. Enzyme-Based CO2 Capture for Air Recovery Subsystems (2002). Life Support & Biosphere Science 8:181-189.
Green N M. “A spectrophotometric assay for avidin and biotin based on binding of dyes by avidin” Biochem J (1965) 94:23c-24c.
Guex N, Diemand A, Peitsch M C. Protein Modelling for All Trends Biochem Sci (1999) 24:364-367.
Holmberg A, Blomstergren A, Nord O, Lukacs M, Lundeberg J, Uhlén M. The biotin-streptavidin interaction can be reversibly broken using water at elevated temperatures. Electrophoresis (2005) (3):501-10.
Humphrey W, Dalke A, Schulten K. VMD: visual molecular dynamics J Mol Graph (1996) 14:33-38.
Jeyakanthan J, Rangarajan S, Mridula P, Kanaujia S P, Shiro Y, Kuramitsu S, Yokoyama S, Sekar K. Observation of a calcium-binding site in the gamma-class carbonic anhydrase from Pyrococcus horikoshii Acta Cryst (2008) D64:1012-1019.
Kay B K, That S, Volgina V V. High-Throughput Biotinylation of Proteins Meth Mol Biol (2009) 498:185-198.
Katz E Y. A chemically modified electrode capable of a spontaneous immobilization of amino compounds due to its functionalization with succinimidyl groups. J. Electroanal. Chem. (1990) 291, 257-260
Khalifah, R G. Carbon dioxide hydration activity of carbonic anhydrase. I. Stop-flow kinetic studies on the native human isoenzymes B and C. J. Biol. Chem. (1971) 246:2561-2573.
Kisker C, Schindelin H, Alber B E, Ferry J G, Rees D C. A left-hand beta-helix revealed by the crystal structure of a carbonic anhydrase from the archaeon Methanosarcina thermophila” EMBO J (1996) 15:2323-2330.
Maren T H. A simplified micromethod for the determination of carbonic anhydrase and its inhibitors. J Pharmacol Exp Ther (1960) 130:26-29.
Repo S, Paldanius T A, Hytönen V P, Nyholm T K, Halling K K, Huuskonen J, Pentikäinen O T, Rissanen K, Slotte J P, Airenne T T, Salminen T A, Kulomaa M S, Johnson M S. Binding properties of HABA-type azo derivatives to avidin and avidin-related protein 4. (2006) Chem Biol. 10:1029-39.
Sasaki Y C, Yasuda K, Suzuki Y, Ishibashi T, Satoh I, Fujiki Y, Ishiwata, S. Two-Dimensional Arrangement of a Functional Protein by Cysteine-Gold Interaction: Enzyme Activity and Characterization of a Protein Monolayer on a Gold Substrate (1997) Biophysical Journal 72:1842-1848
Trachtenberg M C. Novel enzyme compositions for removing carbon dioxide from a mixed gas (2008) US Patent Application 20080003662
Weber P C, Ohlendorf D H, Wendoloski J J, Salemme F R. “Structural Origins of High Affinity Biotin Binding to Streptavidin” Science (1989) 243:85-88.
Weber P C, Wendoloski J J, Pantoliano M W, Salemme F R. Crystallographic and Thermodynamic Comparison of Natural and Synthetic Ligands Bound to Streptavidin J. Am. Chem. SOC. (1992) 114, 3197-3200
Weber P C, Pantoliano M W, Simons, D M, Salemme F R. Structure-Based Design of Synthetic Azobenzene Ligands for Streptavidin J. Am. Chem. SOC, (1994) 116, 2717-2724
Zimmerman S A, Tomb J F, Ferry J G. Characterization of CamH from Methanosarcina thermophila, Founding Member of a Subclass of the γ Class of Carbonic Anhydrases J. Bacteriol. (2010) 192(5):1353-1360

1. An engineered gamma carbonic anhydrase enzyme (gCA) polypeptide comprising
residues 1-213 of Table 1, Sequence 1 (SEQ ID NO: 8) or a sequence greater than 90% identical thereto,
residues 1-173 of Table 1, Sequence 4 (SEQ ID NO: 11) or a sequence greater than 90% identical thereto, or
residues 1-181 of Table 1, Sequence 5 (SEQ ID NO: 12) or a sequence greater than 90% identical thereto.

2. (canceled)

3. The engineered gCA polypeptide of claim 1, having the sequence of Table 1, Sequence 1 (SEQ ID NO: 8) or a sequence greater than 90% identical thereto.

4. The engineered gCA polypeptide of claim 1, having the sequence of Table 1, Sequence 2 (SEQ ID NO: 9) or a sequence greater than 90% identical thereto.

5. The engineered gCA polypeptide of claim 1, having the sequence of Table 1, Sequence 3 (SEQ ID NO: 10) or a sequence greater than 90% identical thereto.

6. The engineered gCA polypeptide of claim 1, having the sequence of Table 1, Sequence 4 (SEQ ID NO: 11) or a sequence greater than 90% identical thereto.

7. The engineered gCA polypeptide of claim 1, having the sequence of Table 1, Sequence 5 (SEQ ID NO: 12) or a sequence greater than 90% identical thereto.

8. The engineered gCA polypeptide of claim 1, having the sequence of Table 1, Sequence 6 (SEQ ID NO: 13) or a sequence greater than 90% identical thereto.

9. The engineered gCA polypeptide of claim 1, having the sequence of Table 1, Sequence 7 (SEQ ID NO: 14) or a sequence greater than 90% identical thereto.

10. The engineered gCA polypeptide of claim 1, having the sequence of Table 1, Sequence 8 (SEQ ID NO: 15) or a sequence greater than 90% identical thereto.

11. An engineered gCA polypeptide comprising a polypeptide sequence of the form A(BDBD)ᵥBC,
wherein v is 0 or 1,
wherein A is a sequence of Amino Terminus Sequence List A that is selected from the group consisting of
no amino acid,
HₙXₘ, wherein X is any amino acid and m ranges from 0 to 20 and n ranges from 0 to 7 or from 4 to 7 (SEQ ID NO: 52), and
LERAPGGLNDIFEAQKIEWHEXᵣ (SEQ ID NO: 49), wherein each amino acid of the Xᵣ subsequence is independently selected as any amino acid and r ranges from 0 to 7 or from 4 to 7,
wherein B is a sequence of Sequence List B that is selected from the group consisting of SEQUENCES 9 through 41 of Table 2,
wherein C is a sequence of Carboxy Terminus Sequence List C that is selected from the group consisting of
no amino acid,
XₚH_{q}, wherein X is any amino acid and p ranges from 0 to 20 and q ranges from 0 to 7 or from 4 to 7 (SEQ ID NO: 53), and
XₛLERAPGGLNDIFEAQKIEWHE (SEQ ID NO: 50), wherein each amino acid of the Xₛ subsequence is independently selected as any amino acid and s ranges from 0 to 7 or from 4 to 7,
wherein D is a sequence of Sequence List D that is GₐS_{b}G_{c}S_{d} (SEQ ID NO: 51), wherein a, b, c, and d each independently range from 0 to 4.

12. A trimeric gCA construct comprising
a first engineered gCA polypeptide of claim 11, a second engineered gCA polypeptide of claim 11, and a third engineered gCA polypeptide of claim 11, each having a sequence of form ABC,
wherein the first engineered gCA polypeptide is bound through a zinc atom to the second engineered gCA polypeptide,
wherein the second engineered gCA polypeptide is bound through a zinc atom to the third engineered gCA polypeptide, and
wherein the third engineered gCA polypeptide is bound through a zinc atom to the first engineered gCA polypeptide.

13. A trimeric trigonal scaffold unit, comprising:
the trimeric gCA construct of claim 12, wherein each engineered gCA polypeptide further comprises a specific binding site comprising a pair of bound biotin or biotin derivative groups; and
three streptavidin tetramers, wherein each streptavidin tetramer has a top pair of biotin binding sites and a bottom pair of biotin binding sites,
wherein the pair of bound biotin or biotin derivative groups of each engineered gCA polypeptide is bound to the top pair of biotin binding sites of the streptavidin tetramer, so that the bottom pairs of biotin binding sites of the three streptavidin tetramers are in a trigonal arrangement.

14. The trimeric trigonal scaffold unit of claim 13, where an avidin tetramer is substituted for the streptavidin tetramer.

15. A single chain gCA construct comprising the engineered gCA polypeptide of claim 11, having a sequence of form ABDBDBC.

16. A single chain trigonal scaffold unit, comprising
the single chain gCA construct of claim 15, wherein each B sequence of the engineered gCA polypeptide further comprises a specific binding site comprising a pair of bound biotin or biotin derivative groups; and
three streptavidin tetramers, wherein each streptavidin tetramer has a top pair of biotin binding sites and a bottom pair of biotin binding sites,
wherein the pair of bound biotin or biotin derivative groups of each B sequence of the engineered gCA polypeptide is bound to the top pair of biotin binding sites of the streptavidin tetramer, so that the bottom pairs of biotin binding sites of the three streptavidin tetramers are in a trigonal arrangement.

17. The single chain trigonal scaffold unit of claim 16,
wherein the specific binding site comprises a pair of cysteine substitutions,
wherein the bound biotin or biotin derivative group is bound to the cysteine substitution,
wherein the pair of bound biotin or biotin derivative groups are located complimentary to a pair of biotin binding sites on streptavidin.

18. (canceled)

19. A di-biotin linked 2D hexagonal lattice, comprising multiple single chain trigonal scaffold units of claim 16,
wherein each single chain trigonal scaffold unit is connected to another single chain trigonal scaffold unit by a pair of bi-functional crosslinking agents,
wherein each bi-functional crosslinking agent comprises two binding groups,
wherein each binding group of the bi-functional crosslinking agent binds to the bottom pair of biotin binding sites in the streptavidin, and
wherein the binding group is biotin, a biotin derivative, desthiobiotin, iminobiotin, HABA (4′-hydroxyazobenzene-2-carboxylic acid), a HABA derivative, or an amino acid sequence comprising WSHPNFEK (SEQ ID NO: 54) or a sequence about 90% or greater identical thereto.

20. A surface immobilized protein construct, comprising:
a first engineered gCA polypeptide of claim 15 having a biotin group covalently bonded to a sequence inserted at or near its amino terminus or carboxy terminus;
a second engineered gCA polypeptide of claim 15 having a biotin group covalently bonded to a sequence inserted at or near its amino terminus or carboxy terminus;
a streptavidin tetramer having a first top and a second top biotin binding site and a first bottom and a second bottom biotin binding site; and
two biotin groups bound to a surface,
wherein the biotin group of the first engineered gCA polypeptide is bound to the first top biotin binding site of the streptavidin tetramer,
wherein the biotin group of the second engineered gCA polypeptide is bound to the second top biotin binding site of the streptavidin tetramer,
wherein the first bottom and second bottom biotin binding sites are bound to the two biotin groups bound to the surface.

21.-22. (canceled)

23. The single chain gCA construct of claim 15,
wherein sequence A is HₙXₘ (SEQ ID NO: 52), optionally
bound to a metal, or LERAPGGLNDIFEAQKIEWHEXᵣ (SEQ ID NO: 49) and wherein sequence C is XₚH_{q} (SEQ ID NO: 53), optionally bound to a metal, or XₛLERAPGGLNDIFEAQKIEWHE (SEQ ID NO: 50).

24.-27. (canceled)

28. A two-dimensional nanostructure, comprising:
the di-biotin linked 2D hexagonal lattice on a fluid layer coated on a substrate,
wherein each single chain gCA construct has a terminus,
wherein the terminus of the single polypeptide chain of the single chain gCA construct comprises a polyhistidine,
the fluid layer comprising a metal chelate,
wherein the polyhistidine is bound to the metal chelate.

29. The two-dimensional nanostructure of claim 28, wherein the single chain gCA construct has a stable tertiary structure at a temperature of about 70° C. or greater.

30.-31. (canceled)

32. A method, comprising:
introducing a nucleotide sequence coding for an engineered gCA amino acid sequence having an Amino Terminal Biotinylation Sequence or a Carboxy Terminus Biotinylation Sequence into a host organism,
culturing the host organism,
lysing the host organism to release the engineered gCA amino acid sequence into a first solution,
biotinylating the engineered gCA amino acid sequence,
contacting the first solution with a substrate functionalized with an engineered avidin at a first pH, so that the biotinylated gCA amino acid sequence binds to the engineered avidin, and
contacting the substrate with the engineered avidin with a second solution at a second pH, so that the engineered avidin releases the biotinylated gCA amino acid sequence in a purified form,
wherein the Amino Terminal Biotinylation Sequence is LERAPGGLNDIFEAQKIEWHEXᵣ (SEQ ID NO: 49), wherein each amino acid of the Xᵣ subsequence is independently selected as any amino acid and r ranges from 0 to 7 or from 4 to 7, and
wherein the Carboxy Terminal Biotinylation Sequence is. X_{S}LERAPGGLNDIFEAQKIEWHE (SEQ ID NO: 50), wherein each amino acid of the X_{S} subsequence is independently selected as any amino acid and s ranges from 0 to 7 or from 4 to 7.

33. (canceled)


SEQUENCE LISTING (54 sequences)

SEQ ID NO: 1 - protein, 6 aa, Artificial Sequence
  Description of Artificial Sequence Synthetic 6xHis tag
HHHHHH

SEQ ID NO: 2 - protein, 21 aa, Artificial Sequence
  Description of Artificial Sequence Synthetic peptide
LERAPGGLNDIFEAQKIEWHE

SEQ ID NO: 3 - DNA, 716 bp, Artificial Sequence
  Description of Artificial Sequence Synthetic polynucleotide
gaaggagatatacatatgcaagagattaccgttgacgaatttagcaatatccgtgaaaacccggttaccccgtggaacccggaaccgagcgcccccggttattgacccgaccgcctatattgacccggaagcaagcgtgattggtgaagttacgattggcgcaaatgttatggttagcccgatggcgagcattcgcagcgatgaaggtatgccgatttttgtgggttgtcgtagcaatgttcaagatggtgttgtcctgcacgcactggaaacgattaatgaagaaggtgaaccgattgaagataatattgttgaagttgatggcaaagaatacgcagtttatattggtaataatgttagcctggcccatcagagccaagtccacggtccggccgcaggcgatgatacgtttattggcatgcaagcgttcgtttttaaaagcaaagtgggtaataatgcagttctggaaccgcgtagcgcagcgattggtgtcacgatcccggatggtcgctatatcccggccggtatggtcgttaccagccaagcagaagcagacaaactgccggaagtcaccgatgattacgcctatagccataccaatgaagccgttgtttgtgtgaatgttcatctggcggaaggttacaaagaaacgattgaaggccgtcatcaccaccacccaccactaagacccagctttcttgtacaaagtggtcccc

SEQ ID NO: 4 - protein, 6 aa, Artificial Sequence
  Description of Artificial Sequence Synthetic peptide
GGSGGG

SEQ ID NO: 5 - DNA, 2029 bp, Artificial Sequence
  Description of Artificial Sequence Synthetic polynucleotide
ggggacaagtttgtacaaaaaagcaggcaccgaaggagatatacatatggatgaatttagcaatatccgcgaaaatccggtgaccccgtggaatccggaaccgagcgcccccggttattgatccgacggcatacatcgacccggaagccagcgtgattggtgaagttaccatcggcgccaatgttatggtcagcccgatggcgagcatccgcagcgatgaaggcatgccgatctttgtgggctgtcgtagcaatgtgcaggatggcgttgttctgcacgcgctggaaaccattaatgaagaaggcgaaccgattgaagacaatattgttgaagtggacggtaaggaatatgcagtgtacatcggtaacaacgtcagcctggcccatcagagccaagtccatggtccggccgccgtgggcgatgataccattggcatgcaagcgttcgtgtttaaaagcaaagttggcaataatgcagttctggaaccgcgcagcgcggcgatcggcgtgaccattccggatggtcgttacatcccggccggcatggtggtcaccagccaagcggaggccgataaactgccggaagtcaccgatgactatgcctatagccacaccaatgaggccgtcgtgtgcgtgaacgttcatctggccgaaggttataaagaaacgggtggtagcggcggcggcgatgaatttagcaatatccgcgaaaatccggtgaccccgtggaatccggagccgagcgcaccggttarrgatccgaccgcatatattgatccggaggccagcgttatcggcgaagttacgatcgcgaatgttatggtgagcccgatggcgagcattcgcagcgatgagggtatgccgatttttgtgggctgccgtagcaatgtgcaagatggtgtggtcctgcacgcactggagacgattaacgaggaaggtgaaccgatcgaggacaacattgtcgaagtggacggtaaggagtatgcggtgtatatcggcaacaacgttagcctggcccaccagagccaggtgcacggcccggcagcagtgggcgatgacacgtttattggcatgcaggcgttcgttttcaaaagcaaagttggcaataacgcagttctggaaccgcgtagcgcagcgattggcgttaccatcccggatggccgttatatcccggccggtatggtcgttacgcaggcggaagcagataaactgccggaagttaccgatgactatgcctatagccataccaatgaggcagttgtttgtgtcaatgtccatctggcggaaggctacaaagaaacgggtggtagcggtggcggtgatgaattcagcaacatccgtgaaaacccggtgaccccgtggaacccggaaccgagcgcgccggtcattgatccgaccgcatatatcgatccggaggcaagcgtcattggcgaagttacgattggcgccaacgtgatggtcagcccgatggccagcatccgcagcgatgaaggcatgccgatttttgttggttgccgtagcaacgttcaggatggcgtggtcctgcacgcactggaaaccattaacgaagaagagccgattgaagataacatcgttgaggtcgacggtaaagaatatgccgtgtatatcggcaacaacgttagcctggcccatcaaagccaagttcatggtccggccgcggttggtgatgacacgttcattggcatgcaggcgtttgtgtttaagagcaaagtgggtaataatgccgttctggagccgcgcagcgccgcaatcggcgtcaccatcccggacggtcgctacattccggcaggcatggtcgtgaccagccaagccgaagcggacaaactgccggaagtcaccgatgattagcatacagccacaccaacgaggcggtcgtgtgtgttaatgtgcatctggcggaaggttataaagaaacgattgaaggccgtcatcaccaccatcattgaacccagctttcttgtacaaagtggtgatgatccggctgctaacaaagcccgaaaggaagctga

SEQ ID NO: 6 - DNA, 2068 bp, Artificial Sequence
  Description of Artificial Sequence Synthetic polynucleotide
cgatgcgtccggcgtagaggatcgagatctcgatcccgcgaaattaatacgactcactatagggagaccacaacggtttccctctagatcacaagtttgtacaaaaaagcaggcaccgaaggagatatacatatggatgaatttagcaatattcgcgaaaacccggttaccccgtggaacccggaaccgagcgcgccggttatcgacccgacggcctacattgatccggaggcaagcgtgattggtgaagtgacgattggtgcaaatgtcatggtgagcccgatggcgagcattcgtagcgatgaaggtatgccgattttcgttggttgtcgtagcaatgttcaagatggtgttgttctgcacgccctggaaaccattaatgaagaaggtgagccgattgaagacaacatcgttgaagttgatggtaaagaatacgcggtttatatcggcaacaacgtcagcctggcacatcagagccaagttcatggtccggcagcagtgggcgatgatacgattggtatgcaagcattcgtttttaaaagcaaagttggtaataatgcagttctggaaccgcgcagcgcagcaattggtgttaccattccggatggtcgttatatcccggccggtatggtggtgacgagccaggcggaagcagataaactgccggaagtgacggatgattatgcctatagccataccaatgaagcagtcgtgtgtgttaacgtgcacctggccgaaggttacaaagaaacgggcggtggtagcggtggcggcgatgaatttagcaataccgtgaaaacccggttacccgtggaatccggaaccgagcgcaccggttattgatccgacggcatatatcgacccggaggcaagcgtgattggcgaagttacgggcgcaaatgtgatggttagcccgatggccagcattcgtagcgatgaaggcatgccgatttttgtggctgccgcagcaatgttcaagatggtgttgtcctgcacgcactggagaccatcaatgaagaaggtgaaccgattgaagataacatcgtcgaagttgacggcaaagaatatgcggtgtatattggcaataatgtcagcctggcacatcaaagccaagttcacggtccggcagcagtgggcgatgatacctttattggcatgcaagcgtttgttttcaaaagcaaagtcggcaataatgcagttctggaaccgcgcgcagcgcagcgattggcgtcacgatcccggatggtcgttatattccggccggcatggtggtgagccaggcagaagcagataaactgccggaagtgaccgatgactatgcctatagccatacgaacgaagccgttgtttgcgtgaacgtgcacctggcagaaggctacaaagaaaccggtggtggcagcggcggcggtgatgaattcagcaatattcgcgaaaatccggtcaccccgtggaatccggaaccgagcgccccggtcattgacccgacggcatatattgatccggaagcaagcgttattggtgaagttacgattggtgcaaacgtgatggtgagcccgatggcgagcattcgcagcgatgagggcatgccgatttttgtgggcgatcgcagcaatgttcaagatggtgttgtcctgcacgccctggaaaccatcaatgaaggcgaaccgattgaagacaatattgtggaagtcgatggtaaagaatacgcagtctatattggtaataatgttagcctggcacatcagagccaagtccacggtccggccgcagtgggtgatgacagtttattggtatgcaagcatttgtgtttaaaagcaaagtcggtaacaatgcagttctggaaccgcgcagcgcagcaatcggcgttacgatcccggatggccgttatatcccggcgggtatggtggttacgagccaagcagaagcggataaactgccggaagttacggatgattatgcctatagccatacgaacgaagcggttgtctacgttaacgtgcatctggcggagggttacaaagaaacgattgagggtcatcatcaccatcatcattgaaacccagctttc

SEQ ID NO: 7 - protein, 5 aa, Artificial Sequence
  Description of Artificial Sequence Synthetic peptide
GSGGS

SEQ ID NO: 8 - protein, 223 aa, Artificial Sequence
  Description of Artificial Sequence Synthetic polypeptide
MQEITVDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDIFIGMQAFVFKSKVGNNAVLEPRSAAIGVTIPDGRYIPAGMVVTSQAEADKLPEVTDDYAYSHTNEAVVCVNVHLAEGYKETIEGRHHHHHH

SEQ ID NO: 9 - protein, 644 aa, Artificial Sequence
  Description of Artificial Sequence Synthetic polypeptide
MDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVCVNVHLAEGYKETGGSGGGDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVCVNVHLAEGYKETGGSGGGDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVCVNVHLAEGYKETIEGRHHHHHH

SEQ ID NO: 10 - protein, 644 aa, Artificial Sequence
  Description of Artificial Sequence Synthetic polypeptide
MDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVCVNVHLAEGYKETGGSGGGDEFSNIRENPVTPWNPEPSAPVIDPTAYIDPEASVIGEVTIGANVMVSPMASIRSDEGMPIFVGCRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVCVNVHLAEGYKETGGSGGGDEFSNIRENPVTPWNPEPSAPVIDPTAYIDPEASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNAVLEPRSAAIGVTIPDGRYIPAGMVVTSQAEADKLPEVTDDYAYSHTNEAVVYVNVHLAEGYKETIEGRHHHHHH

SEQ ID NO: 11 - protein, 181 aa, Artificial Sequence
  Description of Artificial Sequence Synthetic polypeptide
MAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGGHHHHHH

SEQ ID NO: 12 - protein, 181 aa, Artificial Sequence
  Description of Artificial Sequence Synthetic polypeptide
MAIYEINGKKPRIHPSAFVDENAVVIGDVVLEEKTSVWPSAVLRGDIEQIYVGCYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNACIYVELAEKHIKGRKRIGGHHHHHH

SEQ ID NO: 13 - protein, 535 aa, Artificial Sequence
  Description of Artificial Sequence Synthetic polypeptide
MAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGGHHHHHH

SEQ ID NO: 14 - protein, 535 aa, Artificial Sequence
  Description of Artificial Sequence Synthetic polypeptide
MAIYEINGKKPRIHPSAFVDENAVVIGDVVLEEKTSVWPSAVLRGDIEQIYVGCYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNACIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGGHHHHHH

SEQ ID NO: 15 - protein, 550 aa, Artificial Sequence
  Description of Artificial Sequence Synthetic polypeptide
MAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGSGGSAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRIGGLERAPGGLNDIFEAQKIEWHE

SEQ ID NO: 16 - protein, 173 aa, Pyrococcus horikoshii
MAIYEINGKKPRIHPSAFVDENAVVIGDVVLEEKTSVWPSAVLRGDIEQIYVGKYSNVQDNVSIHTSHGYPTEIGEYVTIGHNAMVHGAKVGNYVIIGISSVILDGAKIGDHVIIGAGAVVPPNKEIPDYSLVLGVPGKVVRQLTEEEIEWTKKNAEIYVELAEKHIKGRKRI

SEQ ID NO: 17 - protein, 214 aa, Methanosarcina thermophila
MQEITVDEFSNIRENPVTPWNPEPSAPVIDPTAYIDPEASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 18 - protein, 229 aa, Thermosynechococcus elongates
MGSSHHHHHHSSGLVPRGSHMAVQSYAAPPTPWSRDLAEPEIAPTAYVHSFSNLIGDVRIKDYVHIAPGTSIRADEGTPFHIGSRTNIQDGVVIHGLQQGRVIGDDGQEYSVWIGDNVSITHMALIHGPAYIGDGCFIGFRSTVFNARVGAGCVVMMHVLIQDVEIPPGKYVPSGMVITTQQQADRLPNVEESDIHFAQHVVGINEALLSGYQCAENIACIAPIRNELQ

SEQ ID NO: 19 - protein, 187 aa, Methanosarcina thermophila
MKRNFKMHLPNPHKQHPKVSKRAWISETALIIGNVSIADDVFVGPNAVLRADEPGSSITVHRGCNVQDNVVVHSLSHSEVLIGKNTSLAHSCIVHGPCRIGEDCFIGFGAVVFDCNIGKDTLVLHKSIVRGVDISSGRMVPDGTVITRQDCADALEDITKDLTEFKRSVVKANIDLVEGYIRLREES

SEQ ID NO: 20 - protein, 169 aa, Sulfolobus solfataricus
MPIEEYLGKTPKVSQKAYIHPTSYIIGDVEIGDLTSIWHYVVIRGDNDSIRIGKESNVQENTTIHTDYGYPVEIGDKVTIGHNAVIHGAKVSSHVIVGMGAILLNGSQVKEYSIIGAGSVVTQGTVIPPYSVAVGVPAKVIKKLREDEILIIDENAEEYLKHTRRLLKL

SEQ ID NO: 21 - protein, 151 aa, Methanothermobacter thermautotrophicus
MGFRVLDGARIVGDVRIGDGSSVWYNAVLRGDLEPIEIGRCSNIQDNCVVHTSRGYPVRVGDCVSVGHAAVLHGCIVADNVLIGMNSTILNGAVIGENSIVGAGAVITSGKEFPPGSLIIGTPARAVRELSDEEIESIRDNARRYALLARE

SEQ ID NO: 22 - protein, 167 aa, Dictyoglomus thermophilum
MLRPFEENLPQIEGEVYISGSAVVIGKVTLKKGVNIWDFAVIRGDLDSIFIDEYTNIQENVVIHVDEGKPVYIGKYVTVGHSAVLHGCKIEDNTLVGMGAIILDDAVIGKNSIIGAGTLIPQGKEIPEGSVVIGVPGKIVRSVTEEEILHIKKNAELYYYLSKKYWR

SEQ ID NO: 23 - protein, 214 aa, Methanosaeta thermophila
MSEKSIWPAASVPEPPDLPYPSERSDWEALWCEPVVDETAWVSPGAVLIGRVVLKRESSVWYGCVLRGDESYIEVGEKSNIQDCSVLHVEPDTPCIIGDHVTLGHRVTVHASHIEDWAMVGIGATVLSGSVVGSGAIVAAGALVLEGTKVPPETLWAGVPAREIRKVTPELRERVISTNRQYANRAAMYLHREKLLAKGRGQQGSHQHSDNILL

SEQ ID NO: 24 - protein, 177 aa, Thermosynechococcus elongatus
MVITAPSAFWPPVASDRAAFIAPNATLVGDVRLGEGCSIWYGAVLRGDVTYIEIGAHTNVQDGAILHGDPGQPTILGEEVTVGHRAVIHGATVEDGCLIGIGAVVLNGVRVGAGSIVGAGAVVSKDVPPRSLVLGIPAKVVREVSDTEAADLRQHARKYEQLAQVHKGTGRNLGFSA

SEQ ID NO: 25 - protein, 189 aa, Rhodothermus marinus
MIRDFLGAYPRFDATNFIAPNAVVIGDVTLEPYASIWYGAVVRADVNWIRIGEASNIQDGAIIHVTRGTAPTLIGPRVTVGHGAVLHGCTVEENVLIGIGAVVLDGAVIGRDTIIGARALVPPGMKVPPRSLVLGVPGRVVRTLTDEEVAGIARYAQNYLEYSAIYRGEVQPERNPFYDPSETPDGHSG

SEQ ID NO: 26 - protein, 185 aa, Thermoanaerobacter sp.
MIIKEYKSMKPKIDDEAYIAETAEVIGDVEIKKDANIWYGAVLRGDIDKIVVGEGTNIQDNCVVHVTEGHPCYIGNYCTIGHGAIVHACKIGNNVLIGMGTIILDDAEIGDNCIIGAGSLVTGGKKIPEGSLAFGNPAKVIRKLTQEEIENIHRSYEHYVELAKLHFSNFGKLTVYNKSNIIENS

SEQ ID NO: 27 - protein, 172 aa, Spirochaeta thermophila
MLHAIGERVPRMDETAFVAWNAEVCGSVELGPHASVWFGASVRADIAPITIGAHTNVQDNASVHVDVDLPVVIGSYVTIGHNAVIHGCTIGDGSLIGMGAVVLSGAVIGEESLVGAGALVTEGKEFPPRSLILGSPARVVRSLTDEEVARIRRNALLYAELARSARQEYREV

SEQ ID NO: 28 - protein, 185 aa, Thermoanaerobacter tengcongensis
MIIKEYKGIKPQIDEEAYIAETAEIIGDVEIKKNVNIWYGAVLRGDVDKIVVEEGTNIQDNCVVHVTDGHPCYIGKYCTIGHGAIVHACKVGNNVLIGMGAIILDDAEIGDNCIIGAGALVTGGKKIPPGSLVIGSPAKVVRQLTEEEIESIHKSYEHYVELAKLHFSEFGQLTVYNKSNIIENS

SEQ ID NO: 29 - protein, 179 aa, Thermaerobacter marianensis
MSLYRLGAATPRIAPTAYVAPGARVVGRVVLDEHSSIWFGAVLRGDLDEIRIGAGSNVQDNAVLHVNAGEPCWIGRDVTIGHGAIVHGCTIEDECLIGMGAVVLSRARIGRGSLVGAGAVVPEGKVIPPGSLVLGVPARVVRALTPEEQAEIRAAAARYRENARRFATELTALEAHSQW

SEQ ID NO: 30 - protein, 230 aa, Thermus thermophilus
MSVYRFEDKTPAVHPTAFIAPGAYVVGAVEVGEGASIWFGAVVRGDLERVVVGPGTNVQDGAVLHADPGFPCLLGPEVTVGHRAVVHGAVVEEGALVGMGAVVLNGARIGKNAVVGAGAVVPPGMEVPEGRLALGVPARVVRPIDPPGNAPRYRALAERYRKALFPVAPPRRYRLTLRGQDALNPFSEVHLRLKRTRREALEVLRRAAQGFPLDPEEALPLLAEGLLAPE

SEQ ID NO: 31 - protein, 230 aa, Thermus thermophilus; identical to SEQ ID NO: 30

SEQ ID NO: 32 - protein, 176 aa, Hydrogenobacter thermophilus
MALVKPYRGVYPQIHPSVYLSENVVIVGDVHIGEDSSIWFGTVIRGDVNYIRIGKRTNIQDNCVVHVTHNTYPTIVGDGVTVGHRVVLHGCTLGNYVLVGMGAVVMDGVEVEDYVLIGAGALLTPGKRIPSGVLVAGVPAKIIRDLKPEEVELIKRSAENYVAYKNSYMSADAQKR

SEQ ID NO: 33 - protein, 234 aa, Meiothermus silvanus
MSVYRLEDWEPKIHPSAFVAPEAVVIGQVEVGEGASLWFGAVARGDAEKIVIGAGTNVQDGAILHADPGDPCLLGKNVTVGHRAVVHGATVEDGALIGIGAVVLNKAKIGKGAVVGAGALVPMGMEVPGGTLVVGVPAKVKGPAEKPTHAPRYRALAQRYKGGLYEVKAMPRYRLTLRGQDALNPFSDLHLSLKREHPQAIGLLRSVAEGKLEGLEGNSPILQLLLREGLLSQS

SEQ ID NO: 34 - protein, 178 aa, Thermomicrobium roseum
MRPLVIPYRGKQPQLAPDVFVAPTAVVIGDVVVGSRSSLWFGVVLRGDIGPIRIGQRVNLQEGVIVHLDEGFPVVIEDDVTIGHGAIVHGAQIAAGAQIGMGAILLTGSRVGAGAIVAAGALVPEGMEVPAGTVAVGIPARIRREVTTEERAELLERAQRYAQRGEEFRRLLAGGGEA

SEQ ID NO: 35 - protein, 185 aa, Thermoanaerobacter mathranii
MIIKEYKGMKPKIDDEAYIAETAEVIGDVEIKKDANIWYGAVLRGDIDKIVVGEGTNIQDNCVVHVTEGHPCYIGNYCTIGHGAIVHACKIGNSVLIGMGAIILDDAEIGDNCIIGAGSLVTGGKKIPEGSLAFGNPAKVIRKLTQEEIENIHRSYEHYVELAKLHFSNFGQLTVYNKSNIIENS

SEQ ID NO: 36 - protein, 175 aa, Thermobispora bispora
MPYIAELDGGATPDIHPEAWIAPGAVVVGKVRLGRASNVWYGSVLRGDDEWIEVGAECNIQDLCCLHADPGEPAILKDRVSLGHRAMVHGARVEQGALIGIGAVVLGGAVIGAGSLIAAGAVVTPGTKIPAGVLVAGVPGRIIRELTDADRASFAKTPDRYVAKARRHAAANRLR

SEQ ID NO: 37 - protein, 185 aa, Thermoanaerobacter italicus
MIIKEYKGMKPKIDDEAYIAETAEVIGDVEIKKDVNIWYGAVLRGDIDKIVVGEGTNIQDNCVVHVTEGHPCYIGNYCTIGHGAILHACKIGNNVLIGMGAIILDDAEIGDNCIIGAGSLVTGGKKIPEGSLAFGNPAKVIRKLTQEEIENIRHSYELYVELAKLHFSNFGQLTVYNKSNIIENS

SEQ ID NO: 38 - protein, 205 aa, Thermobifida fusca
MGDCARAWTVSVIFAVRTCLWVGGDAMSGSGERPHIGSAEFGEPTIHPDAWIAPGAVVVGRVRIGAHSSVWYGSVLRADTEDIIVGERCNIQDLCCLHADPGEPAILGNGVSLGHKAMVHGAVVEDGALIGINAVVLGGATVEAGALVAAGALVPPGRRVPAGTLWAGVPGKVIRELTDAERENLVGTAERYVGYAAQHRGVTWR

SEQ ID NO: 39 - protein, 180 aa, Thermocrinis albus
MPIVRPYGDRTPKIHPTVFLAENAVVIGDVEIGEDSSVWYGAVIRGDVNWIRIGKRTNIQDNTVVHVTHQRYPTWIGDYVTVGHSVILHGCKIGNYVLVGMGAVVMDGVEVEDYVLIGAGALLTPHKKFPSGVLVAGVPARVVRDLREEEVEMIKNSAENYVRYKEAYLSSYAQGQQERS

SEQ ID NO: 40 - protein, 184 aa, Thermoanaerobacter tengcongensis
MIREDIFGNYPQIAHSAYVDDTAILIGNIVVGENVYIGPNVVIRADEVDENYRVGKIVIKDKAAIYDGANINTTGASEITIGEGTVISNGVIIKGECHIGNYCSINVKSIIFNSYIGDNCYVGISAVLENVKMPENTMVESGVFLREDNIASLIKPVPEGKINIAGKITLSNKVLINWYKLSGY

SEQ ID NO: 41 - protein, 173 aa, Thermanaerovibrio acidaminovorans
MERENLLAFEGVMPQVDPEAYVAPTACLIGNVKVGKGASVWHGAVLRGDINRIEIGDRSNIQDGCIVHVTDQLPVVVEEDVTVGHGAILHGCTIKRGCLIAMRATVLDGAVVGEGSVIAAGAIVPEGAVIPPGSVVMGIPGKVVREVREKDREKLAFLSSSYVELSSRYKGRR

SEQ ID NO: 42 - protein, 185 aa, Thermoanaerobacter pseudethanolicus
MIIKEYKSMKPKIDDEAYIAETAEVIGDVEIKKDANIWYGAVLRGDIDKIVVGEGTNIQDNCVVHVTEGHPCYIGNYCTIGHGAIVHACKIGDNVLIGMGTIILDDAEIGDDCIIGAGSLVTGGKKIPEGSLAFGNPAKVIRKLTQEEIENIHRSYEHYVELAKLHFSNFGKLTVYNKSNIIENS

SEQ ID NO: 43 - protein, 173 aa, Thermoanaerobacterium thermosaccharolyticum
MTLIKGFGKYFPIIDNSALIADSAAIIGRVKIDKDVNIWYGAVIRGDIDEITIGEGTNIQDNCIVHVTEGHPCIIGKHCTIGHNAIIHSAKIGDNVLIGMGAIILDDAVIEDNCIIGAGALVTGGKVIKGGSMVFGNPAKFVRYLNEDEIKSLDLSYRHYIEIAKSHFKKLSN

SEQ ID NO: 44 - protein, 168 aa, Thermosediminibacter oceani
MIQDFKGKRPDIHQSCFIAPTADIIGDVTVGENSSVWHRAVLRGDINSIKIGANSNIQDGTVIHVAEEHPVTIGDYVTVGHSAILHGCTIKDNALIGMGAIVLDGAVVGEGALVGAGSLVPEGKEIPPYSLAIGIPAKVVRQLTREQIEKIKKNAEDYVEWAKEFMQE

SEQ ID NO: 45 - protein, 171 aa, Caldicellulosiruptor kronotskyensis
MIITYKDKTPKIATSAFVAENAVIIGDVEIGENSSVWFGCVLRCEENRIIIGKNTNIQDLTTIHTDHCCSVIIGDNVTVGHNVVLHGCEIGNNVLIGMGTIIMNGSKIGDNCLIGAGSLITQNMVIPPNTLVFGRPAKVIRELTPEEIEKIAISAREYIELSNEYKKIKGY

SEQ ID NO: 46 - protein, 174 aa, Pelotomaculum thermopropionicum
MILPYDGVRPEIDETAFIAPTAVVVGRVEIGPYSSIWYNSVVRGDVDTVVIGACTSIQDGSILHEHAGFPLVIGDRVTVGHRVLLHGCTVEDGAYIGMGAIVLNGARIGAGAVVGAGSLVLQGQEIPPGMLALGSPARVVRPIREDEVDRFLGAVGRYLKMAEKHARTAAGKAR

SEQ ID NO: 47 - protein, 173 aa, Geobacillus thermodenitrificans
MIYPYKGKTPQIAPSAFIADYVTITGDVTIGEETSIWFNTVIRGDVAPTIIGNRVNIQDNSILHQSPNNPLIIEDGVTVGHQVILHSAIVRKHALIGMGSIILDRAEIGEGAFIGAGSLVPPGKKIPPNVLALGRPAKVVRELTEDDFREMERIRREYVEKGQYYKALQQNRS

SEQ ID NO: 48 - protein, 183 aa, Geobacillus thermodenitrificans
MLYLYNGKKPNVHESVFIAPGARVIGDVTVGEESTIWFNAVLRGDEGPITIGARTSIQDNTTCHLYEGSPLVIEDEVTVGHNVVLHGCTIRRRSIIGMGSTILDGAEIGEECIIGANTLIPSGKKIPPRSLVVGSPGQVVRELTDKDLALIQLSIDTYVQKGKEYRKQLTAAESTDKETSKQV

SEQ ID NO: 49 - protein, 28 aa, Artificial Sequence
  Description of Artificial Sequence Synthetic peptide
LERAPGGLNDIFEAQKIEWHEXXXXXXX

SEQ ID NO: 50 - protein, 28 aa, Artificial Sequence
  Description of Artificial Sequence Synthetic peptide
XXXXXXXLERAPGGLNDIFEAQKIEWHE

SEQ ID NO: 51 - protein, 16 aa, Artificial Sequence
  Description of Artificial Sequence Synthetic peptide
GGGGSSSSGGGGSSSS

SEQ ID NO: 52 - protein, 27 aa, Artificial Sequence
  Description of Artificial Sequence Synthetic peptide
HHHHHHHXXXXXXXXXXXXXXXXXXXX

SEQ ID NO: 53 - protein, 27 aa, Artificial Sequence
  Description of Artificial Sequence Synthetic peptide
XXXXXXXXXXXXXXXXXXXXHHHHHHH

SEQ ID NO: 54 - protein, 8 aa, Artificial Sequence
  Description of Artificial Sequence Synthetic peptide
WSHPNFEK